(19) United States
(12) Patent Application Publication
(10) Pub. No.: US 2026/0337702 A1
(43) Pub. Date: Oct. 29, 2026

(54) DUAL CHANNEL DETECTION SYSTEM FOR SIMULTANEOUS QUANTIFICATION OF HEPATITIS B AND DELTA VIRUSES

(71) Applicant: The Trustees of Princeton University, Princeton, NJ (US)

(72) Inventors: Alexander Ploss, Princeton, NJ (US); Yongzhen Liu, Nanjing (CN)

(73) Assignee: The Trustees of Princeton University, Princeton, NJ (US)

(21) Appl. No.: 19/650,938

(22) Filed: Apr. 17, 2026

Related U.S. Application Data
(60) Provisional application No. 63/790,844, filed on Apr. 18, 2025.

(51) Int. Cl. C12Q 1/70; C12Q 1/6818

(52) U.S. Cl. CPC C12Q 1/706; C12Q 1/6818; C12Q 2600/156

(57) ABSTRACT

A method for simultaneous detection and quantification of hepatitis B virus (HBV) DNA and hepatitis delta virus (HDV) RNA in a sample. The method comprises combining the sample with an HBV primer pair, an HDV primer pair, an HBV probe, and an HDV probe in a reaction mixture, performing a one-step reverse transcription quantitative polymerase chain reaction (RT-qPCR) on the reaction mixture, and detecting a first fluorescent signal from the HBV probe and a second fluorescent signal from the HDV probe to simultaneously quantify HBV DNA and HDV RNA in the sample. The HBV primer pair comprises an HBV forward primer and an HBV reverse primer, and the HDV primer pair comprises an HDV forward primer and an HDV reverse primer. The HBV probe and HDV probe each comprise a fluorescent label attached to a 5′ end and a quencher attached to a 3′ end.

### CROSS-REFERENCE TO RELATED APPLICATIONS

This application claims priority to U.S. Application No. 63/790,844, titled DEVELOPMENT OF A DUAL CHANNEL DETECTION SYSTEM FOR PAN-GENOTYPIC SIMULTANEOUS QUANTIFICATION OF HEPATITIS B AND DELTA VIRUSES, filed Apr. 18, 2025, which is hereby incorporated by reference in its entirety.

### STATEMENT REGARDING FEDERALLY SPONSORED RESEARCH OR DEVELOPMENT

This invention was made with government support under Grant numbers AI138797, AI107301, AI146917, and AI153236 awarded by the National Institutes of Health. The government has certain rights in the invention.

### REFERENCE TO AN ELECTRONIC SEQUENCE LISTING

The contents of the electronic sequence listing (Sequence Listing PRIN99103.xml; Size: 6,026 bytes; and Date of Creation: Apr. 16, 2026) is herein incorporated by reference in its entirety.

### FIELD OF INVENTION

The present disclosure relates to molecular diagnostic assays for viral detection, and more particularly to a dual fluorescence channel detection system for pan-genotypic simultaneous quantification of hepatitis B virus DNA and hepatitis delta virus RNA through one-step quantitative polymerase chain reaction.

### BACKGROUND

Hepatitis B virus (HBV) infection remains a major public health problem as the etiological agent for acute and chronic hepatitis B, with approximately 2 billion people exposed to this virus and at least 257 million individuals living with chronic hepatitis B (CHB) worldwide. Long-term HBV infection can lead to ongoing intrahepatic inflammation, activation of liver fibrogenic processes, and eventual progression to decompensated severe liver disease or hepatocellular carcinoma (HCC). Although nucleos(t)ide analogs and pegylated type I interferon are currently available for antiviral therapies, HBV infection is rarely cured and may require lifelong treatment due to the persistence of HBV covalently closed circular DNA (cccDNA), which serves as the major template for HBV gene transcription.

Hepatitis delta virus (HDV) is a small satellite virus of HBV that depends on HBV surface proteins for its virion assembly and release, and thus HDV spread relies on co-infection with HBV. HDV can be established through superinfection of HBV carriers or co-infection when both viruses infect an individual simultaneously. Recent estimates indicate that a significant percentage of the HBsAg-positive population, corresponding to tens of millions of patients, are co-infected with HDV globally. These percentages may be even higher in certain risk groups.

Compared with chronic HBV infection alone, HBV and HDV dual infection is frequently associated with the most severe form of viral hepatitis and accelerated liver disease progression. The rates of HCC and hepatic decompensation are also higher than for HBV mono-infection. Unfortunately, treatment options for HDV infection remain limited, and unlike anti-HBV nucleos(t)ide analog drugs which can repress and control HBV replication by inhibiting HBV polymerase, these drugs do not inhibit HDV replication because HDV uses host enzymes to propagate its genome.

Clinically, presence of HDV antibody in the blood is used to diagnose ongoing or prior HDV infection, while an active infection can only be diagnosed by detection of HDV RNA. However, due to the lack of a reliable and universal HDV RNA quantification assay, the HDV RNA determination system is not well established, and only a fraction of HDV infected patients have been diagnosed due to limited disease awareness and limited access to reliable diagnostic tests. Given that HBV DNA levels in blood are tested as a standard biomarker to evaluate the virological response under anti-HBV treatment, a detection system capable of monitoring both HBV DNA and HDV RNA simultaneously may be useful for monitoring the efficacy of clinical treatment protocols in co-infected patients.

Existing assays for viral nucleic acid quantification typically focus on a single target, quantifying either HBV DNA or HDV RNA alone, thereby limiting the ability to monitor dynamic interactions between HBV and HDV. Furthermore, some assays employ HDV DNA plasmids or cDNA as standards for HDV RNA quantification, raising concerns about the calibration of reverse transcription efficiency. Additionally, both HBV and HDV exhibit substantial genetic heterogeneity across multiple genotypes distributed globally, which may complicate detection using assays designed for specific viral sequences.

Therefore, improved systems and methods for simultaneous detection and quantification of HBV and HDV across diverse viral genotypes are desired.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for simultaneous detection and quantification of hepatitis B virus (HBV) DNA and hepatitis delta virus (HDV) RNA in a sample is provided. The method comprises combining the sample with an HBV primer pair, an HDV primer pair, an HBV probe, and an HDV probe in a reaction mixture, performing a one-step reverse transcription quantitative polymerase chain reaction (RT-qPCR) on the reaction mixture, and detecting a first fluorescent signal from the HBV probe and a second fluorescent signal from the HDV probe to simultaneously quantify HBV DNA and HDV RNA in the sample. The HBV primer pair comprises an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2]. The HDV primer pair comprises an HDV forward primer having an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4]. The HBV probe comprises a first fluorescent label attached to a 5′ end and a first quencher attached to a 3′ end. The HDV probe comprises a second fluorescent label attached to a 5′ end and a second quencher attached to a 3′ end. This method enables simultaneous monitoring of both HBV DNA and HDV RNA in a single reaction, providing a timely and cost-efficient solution for diagnosing and monitoring co-infected patients while eliminating the need for separate assays.

According to other aspects of the present disclosure, the method may include one or more of the following features. The one-step RT-qPCR may be performed in a single reaction tube. Performing the reaction in a single tube cuts time and costs while simplifying the workflow for clinical and research applications.

The first fluorescent label may be carboxyfluorescein (FAM) and the second fluorescent label may be carboxytetramethylrhodamine (TAMRA). These fluorophores provide robust target specificity with no carryover or contaminated fluorescence signal between detection channels.

The first fluorescent label and the second fluorescent label may emit fluorescence at different wavelengths. Using spectrally distinct fluorescent labels enables simultaneous detection of both viral targets without interference between the two detection channels.

The HBV probe may comprise the nucleotide sequence AGATG+ATTA+GGCAGA+GGTGAAAAAG [SEQ ID NO. 5], wherein+denotes a locked nucleic acid modification. The locked nucleic acid modifications impart heightened structural stability and increase hybridization melt temperature, enabling reliable detection even with shorter sequences in limited conserved regions.

The HDV probe may comprise the nucleotide sequence GGTCC+G+ACCTGGGC+ATCCG [SEQ ID NO. 6], wherein+denotes a locked nucleic acid modification. The locked nucleic acid modifications enhance structural stability and increase hybridization melt temperature for improved detection sensitivity.

The one-step RT-qPCR may comprise a reverse transcription step at a temperature between 40 °C and 70 °C for a period of 5 to 20 minutes, an initial denaturation step at a temperature between 70 °C and 99 °C for a period of less than 5 minutes, and 40 to 60 amplification cycles comprising a denaturation phase and an annealing/extension phase with signal capture. These thermal cycling parameters enable efficient reverse transcription of HDV RNA and subsequent amplification of both targets with high sensitivity.

The sample may comprise a biological sample obtained from a subject. This enables the method to be applied to clinical samples for diagnosing patients and monitoring viral suppression under antiviral therapy.

According to another aspect of the present disclosure, a kit for simultaneous detection and quantification of hepatitis B virus (HBV) DNA and hepatitis delta virus (HDV) RNA is provided. The kit comprises an HBV primer pair comprising an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2], an HDV primer pair comprising an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4], an HBV probe comprising a first fluorescent label attached to a 5′ end and a first quencher attached to a 3′ end, and an HDV probe comprising a second fluorescent label attached to a 5′ end and a second quencher attached to a 3′ end. The first fluorescent label and the second fluorescent label are spectrally distinguishable. This kit provides all necessary components for simultaneous quantification of HBV DNA and HDV RNA, enabling a single quantification process to obtain two readout reports for monitoring both viral loads.

According to other aspects of the present disclosure, the kit may include one or more of the following features. The first fluorescent label may be carboxyfluorescein (FAM) and the second fluorescent label may be carboxytetramethylrhodamine (TAMRA). These fluorophores enable excellent readout specificity with no cross-channel interference even at high viral loads.

The HBV probe may comprise the nucleotide sequence AGATG+ATTA+GGCAGA+GGTGAAAAAG [SEQ ID NO. 5], wherein+denotes a locked nucleic acid modification. The locked nucleic acid modifications provide enhanced structural stability for reliable hybridization across diverse sample conditions.

The HDV probe may comprise the nucleotide sequence GGTCC+G+ACCTGGGC+ATCCG [SEQ ID NO. 6], wherein+denotes a locked nucleic acid modification. The locked nucleic acid modifications increase hybridization melt temperature for improved detection performance.

The kit may further comprise a reverse transcriptase and a DNA polymerase. Including these enzymes enables a complete one-step RT-qPCR workflow for convenient and efficient viral quantification.

According to another aspect of the present disclosure, a dual fluorescence channel detection system for pan-genotypic simultaneous quantification of hepatitis B virus (HBV) DNA and hepatitis delta virus (HDV) RNA is provided. The system comprises a reaction mixture comprising an HBV primer pair targeting a conserved region of the HBV genome, the HBV primer pair comprising an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2], an HDV primer pair targeting a conserved region of the HDV genome, the HDV primer pair comprising an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4], an HBV probe comprising a first fluorescent label and a first quencher, and an HDV probe comprising a second fluorescent label and a second quencher. The system further comprises a quantitative PCR instrument configured to detect the first fluorescent label in a first detection channel and the second fluorescent label in a second detection channel. By targeting conserved regions in the genomes, this system can detect all versions (genotypes) of genetically diverging HBV and HDV, providing a universal detection method advantageous for targeting and identifying different genotypes worldwide.

According to other aspects of the present disclosure, the system may include one or more of the following features. The HBV primer pair may be configured to specifically detect HBV relaxed circular DNA (rcDNA) through over-gap amplification. This design selectively targets infectious HBV with rcDNA genomes while excluding non-infectious HBV particles that only possess short minus-strand DNA, providing accurate quantification of viral load particularly in samples from patients undergoing nucleos(t)ide analog therapy.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following FIGURES.

FIG. 1A is a schematic of the HBV-HDV dual channel one-step quantitative PCR. The DNA and RNA are purified from the samples and HBV DNA and HDV RNA are detected and quantified in a single reaction simultaneously by using HBV and HDV specific primers and probes, respectively.

FIG. 1B shows quantification of serially diluted HBV genomes by (RT-)qPCR in the absence of HDV RNA.

FIG. 1C shows quantification of serially diluted HDV genomes by (RT-)qPCR in the absence of HBV DNA.

FIG. 1D shows serial dilutions of HBV DNA and HDV RNA in the detection samples show the HBV-HDV dual channel quantitative detection system can specifically and simultaneously detect HBV DNA and HDV RNA in a single qPCR.

FIG. 1E shows a representative multicomponent plot to show the fluorescence signal amplification in the dual detection system by using the QUANTSTUDIO™ 7 Flex qPCR machine. The FAM channel was used for HBV DNA, the TAMRA channel was used for HDV RNA and the Rox channel was used as the passive control.

FIG. 1F shows standard curves for HBV DNA (top) and HDV RNA (bottom) quantification. The accuracy of the HBV-HDV dual channel quantitative detection system was validated by analysing the correlations of the quantifications between HBV-HDV dual detection system and the HBV or HDV single detection system through simple linear regression. The R-squared values show the degree of their linear correlation and the P values show the statistical significance. The dashed lines indicate 95% confidence intervals.

FIGS. 1G-1H show the correlations of quantifications between HBV-HDV dual detection system and the HBV (1G) or HDV (1H) single detection system.

FIG. 1I shows characterization of the HBV-HDV dual channel quantitative detection system in the transfection system with mock, HBV mono-transfection or HBV-HDV co-transfection. Data shown as mean±SD of 4 biological replicates.

FIG. 2A shows HBV quantitative PCR primer and probe designment based on the alignment and phylogenetic assay of HBV genomes of different genotypes (A-H). The phylogenetic tree (performed by DNASTAR, the phylogeny was computed using Maximum Likelihood RAxML, order ascending by distance) shows the genome difference among genotypes and for each HBV genotype three representative genomes were used. Sequence alignment was performed under the Clustal W algorithm and non-conserved nucleotides were labelled with different colours. The conserved regions were used for primers and probe designment which were indicated by dashed lines and arrows.

FIG. 2B is a schematic to show the specificity of secreted HBV rcDNA but not HBV pgRNA detection of the HBV and HDV dual channel system. HBV pgRNA was shown with the epsilon structures and the direct repeat elements (DR) in 5′ and 3′ primes. The qPCR primers for HBV DNA in the HBV and HDV dual channel system are shown accordingly based on the HBV genome position. Characterization of the HBV DNA but not HBV pgRNA specific detection by using the supernatants after HBV infectious clone transfection and sera from HBV infected patients.

FIGS. 2C and 2D are charts showing HBV DNA/HBV pgRNA assessments using the supernatants after HBV infectious clone transfection (2C) or sera from HBV infected patients (2D). Data shown as mean±SD of 5 biological replicates (2C) or 6 patient samples (2D). Statistical significance was determined applying an unpaired two-tailed t test.

FIG. 2E shows the pan-genotypic detection of HBV DNA in the HBV and HDV dual channel quantification system is validated by detecting the supernatants after HBV different genotype (A-H) plasmid transfection, respectively. Data shown as mean±SD of at least 3 biological replicates.

FIG. 2F shows HDV quantitative PCR primer and probe design based on the alignment and phylogenetic assay of HDV genomes of different genotypes (1-8). The phylogenetic tree (performed by DNASTAR, the phylogeny was computed using Maximum Likelihood RAxML, order ascending by distance) shows the sequence difference across genotypes and for each HDV genotype three representative genomes were used. Sequence alignment was performed under the Clustal W algorithm and non-conserved nucleotides were labelled with different colours. The conserved regions were used for primers and probe designment which were indicated by dashed lines and arrows.

FIG. 3A shows HBV DNA detection in the supernatants after HBV single transfection, HBV-HDV co-transfection and HDV-HBV preS1 cotransfection.

FIG. 3B shows HDV RNA detection in the supernatants after HBV single transfection, HBV-HDV co-transfection and HDV-HBV preS1 co-transfection.

FIGS. 3C and 3D show HBsAg (3C) and HBeAg (3D) detection in the supernatants after HBV single transfection, HBV-HDV co-transfection and HDV-HBV preS1 co-transfection by ELISA. Statistical significance was determined applying an unpaired two-tailed t-test. n=4.

FIG. 3E shows intracellular HBV core protein and HDV antigen analyzed by western blot after HBV single transfection, HBV-HDV co-transfection and HDV-HBV preS1 co-transfection. Data shown as mean±SD of 2 independent western blot experiment and the quantification was performed 2 times. Statistical significance was determined applying an unpaired two-tailed t-test.

FIG. 4A shows the HepG2-NTCP infectious cell culture model was characterized by flow cytometry and confocal imaging and the cells were used for HDV or HBV mono-infection and HBV & HDV co-infection, respectively.

FIGS. 4B-4C show representative images for immunofluorescence (IF) of HBcAg for the mock control (4B, top), HBV single infection (4B, middle), HDV single (4B, down) and HBV-HDV coinfection (4C) by detecting HBcAg and HDAg, respectively.

FIGS. 4D-4F show quantification of HBeAg levels (4D) by ELISA and HBV DNA (4E) and HDV RNA (4F) copy numbers by (RT-)qPCR in the supernatants at the indicated timepoints. The data are shown as mean±SD of 3 independent experiments.

FIG. 5A shows a schematic of the experimental procedures of HBV-HDV superinfection in human liver chimeric mice.

FIG. 5B shows characterization of the chimeric mouse liver with human hepatocytes by human-specific β2-microglobulin fluorescent immunohistochemistry. Naïve FNRG mouse was used in parallel as a negative control.

FIGS. 5C-5F shows quantification of human albumin (hAlb) (5C), HBsAg (5D) (both by ELISA), HBV DNA (5E) and HDV RNA (5F) in the sera of human liver chimeric mice at the indicated timepoints in mock, HBV mono- and HBV-HDV superinfected mice. BL, base line.

FIG. 5G shows a schematic of the multiplex fluorescent IHC analysis of human β2-macroglobulin, HBc and HDAg.

FIG. 5H shows representative images of multiplex fluorescent IHC of the liver sections 9 weeks post infection. HBc-positive signals are shown as cyan, human β2-macroglobulin as red, and HDAg as yellow.

FIGS. 6A-6B show HBsAg (6A) and HBeAg (6B) levels in the sera from HBV+(n=10) and HBV-HDV dually infected patients (n=4).

FIG. 6C shows HBV genome cloning and sequencing of patient 7. A reference sequence (NCBI access number LC519789.1) was used for the comparison.

FIGS. 6D-6E show quantification of HBV DNA (6D) and HDV RNA (6E) copy numbers by the dual fluorescence channel HBV-HDV quantitative detection system. The dash lines show the low limit of the detection. ND, not detected.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure describes a dual fluorescence channel detection system for pan-genotypic simultaneous quantification of hepatitis B virus (HBV) DNA and hepatitis delta virus (HDV) RNA through a one-step quantitative polymerase chain reaction (qPCR). The dual channel detection system may employ a one-step reverse transcription quantitative PCR (RT-qPCR) system that combines reverse transcriptase, DNA polymerase, dNTPs, and buffer components into a single reaction. For simultaneous quantification of HBV and HDV, the dual channel detection system may utilize spectrally distinguishable fluorescent probes targeting the respective viral genomes, such as carboxyfluorescein (FAM) labeled probes for HBV DNA detection and carboxytetramethylrhodamine (TAMRA) labeled probes for HDV RNA detection. As will be understood, other systems, aside form the FAM-TAMRA system, can be utilized here. Alternative fluorophore pairs that may be utilized in place of the FAM-TAMRA system include, but are not limited to, FAM paired with Cy5, HEX paired with Cy5, VIC paired with TAMRA, FAM paired with Texas Red, Cy3 paired with Cy5, and FAM paired with ROX. Other suitable fluorophore combinations may include JOE paired with Cy5, TET paired with TAMRA, or any combination of spectrally distinguishable fluorescent labels that permit simultaneous detection in separate detection channels without significant spectral overlap or cross-channel interference. The dual channel detection system may exhibit robust target specificity with no carryover or contaminated fluorescence signal between the detection channels, even at high viral loads.

The dual channel detection system may provide pan-genotypic detection capability across diverse HBV and HDV genotypes. HBV exhibits genetic heterogeneity with at least nine classified genotypes (A to I), and HDV exhibits sequence heterogeneity of up to 30% among different genotypes, with eight major clades identified based on phylogenetic classification. The primers and probes of the dual channel detection system may target conserved regions in the HBV and HDV genomes, enabling detection across all genotypes. In some cases, the HBV primer pair may be configured to specifically detect HBV relaxed circular DNA (rcDNA) through over-gap amplification, selectively targeting infectious HBV particles with rcDNA genomes while excluding non-infectious HBV particles that possess only short minus-strand DNA. This design may provide accurate quantification of viral load, particularly in samples from patients undergoing nucleos(t)ide analog therapy.

The dual channel detection system may offer advantages compared to separate single-target assays for HBV DNA and HDV RNA quantification. Through a single quantification process performed in a single reaction tube, the dual channel detection system may provide two readout reports for simultaneous monitoring of HBV and HDV levels. This approach may reduce time and costs while simplifying the workflow for clinical and research applications. The dual channel detection system may provide reliable detection for HBV and HDV in vitro, in human liver chimeric mice, and in patient samples, offering a timely and cost-efficient solution for diagnosing and monitoring co-infected patients.

### [Method for Simultaneous Detection and Quantification]

The method for simultaneous detection and quantification of hepatitis B virus (HBV) DNA and hepatitis delta virus (HDV) RNA in a sample may comprise combining the sample with an HBV primer pair, an HDV primer pair, an HBV probe, and an HDV probe in a reaction mixture. The method may further comprise performing a one-step reverse transcription quantitative polymerase chain reaction (RT-qPCR) on the reaction mixture and detecting a first fluorescent signal from the HBV probe and a second fluorescent signal from the HDV probe to simultaneously quantify HBV DNA and HDV RNA in the sample.

The HBV primer pair may comprise an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2]. The HBV primers may be designed in genomic regions that are highly conserved among different HBV genotypes. The HDV primer pair may comprise an HDV forward primer having an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4]. The HDV primers may be designed in conserved regions of the HDV genome. In some cases, the HDV forward primer may include a degenerate base Y to accommodate a CIT polymorphism between genotype 4 and other genotypes, enabling pan-genotypic detection of HDV.

The HBV probe may comprise a first fluorescent label attached to a 5′ end and a first quencher attached to a 3′ end. The HDV probe may comprise a second fluorescent label attached to a 5′ end and a second quencher attached to a 3′ end. In some cases, the first fluorescent label may be carboxyfluorescein (FAM) and the second fluorescent label may be carboxytetramethylrhodamine (TAMRA). The first fluorescent label and the second fluorescent label may emit fluorescence at different wavelengths, enabling simultaneous detection of both viral targets without interference between the two detection channels. The FAM channel may be used for HBV DNA detection and the TAMRA channel may be used for HDV RNA detection.

Any appropriate quencher may be utilized here. In some examples, the first quencher and the second quencher may each independently be selected from Black Hole Quencher 1 (BHQ-1), Black Hole Quencher 2 (BHQ-2), Black Hole Quencher 3 (BHQ-3), Iowa Black FQ (IB FQ), Iowa Black RQ (IB RQ), Iowa Black RQ-Sp (IB RQSp), TAMRA (when used as a quencher), Dabcyl, Eclipse, or QSY quenchers. The selection of quencher may depend on the emission wavelength of the corresponding fluorescent label to provide efficient fluorescence suppression until probe cleavage occurs during amplification.

In some cases, the HBV probe may comprise the nucleotide sequence AGATG+ATTA+GGCAGA+GGTGAAAAAG [SEQ ID NO. 5], wherein+denotes a locked nucleic acid modification. In some embodiments, the HBV probe may be: FAM—SEQ ID NO. 5—IB FQ. In some cases, the HDV probe may comprise the nucleotide sequence GGTCC+G+ACCTGGGC+ATCCG [SEQ ID NO. 6], wherein+denotes a locked nucleic acid modification. In some embodiments, the HDV probe may be: TAMRA—SEQ ID NO. 6—IB RQSp. The locked nucleic acid modifications may impart heightened structural stability and may increase hybridization melt temperature (Tₘ), enabling reliable detection even with shorter sequences in limited conserved regions.

The one-step RT-qPCR may be performed in a single reaction tube. The reaction mixture may comprise a reverse transcriptase, a DNA polymerase, dNTPs, and buffer components combined into a single reaction. In some cases, the reaction mixture may comprise a Warm Start-activated reverse transcriptase and a Hot Start DNA-dependent DNA polymerase. The primers may be present at a final concentration of approximately 0.4 μM and the probes may be present at a final concentration of approximately 0.2 μM in the single reaction system for HBV and HDV, respectively.

The one-step RT-qPCR may comprise a reverse transcription step at a temperature between 40 °C and 70 °C for a period of 5 to 20 minutes. In some cases, the reverse transcription step may be performed at approximately 55 °C for approximately 10 minutes. During the reverse transcription step, the reverse transcriptase may convert HDV RNA to complementary DNA (cDNA) while the HBV DNA template remains available for subsequent amplification.

The one-step RT-qPCR may further comprise an initial denaturation step at a temperature between 70 °C and 99 °C for a period of less than 5 minutes. In some cases, the initial denaturation step may be performed at approximately 95 °C for approximately 1 minute. The initial denaturation step may activate the Hot Start DNA polymerase and denature the nucleic acid templates.

The one-step RT-qPCR may further comprise 40 to 60 amplification cycles comprising a denaturation phase and an annealing/extension phase with signal capture. In some cases, the amplification cycles may comprise 45 to 50 cycles. Each amplification cycle may comprise a denaturation phase at approximately 95 °C for approximately 15 seconds, followed by an annealing/extension phase at approximately 61 °C for approximately 40 seconds for signal capture. During the annealing/extension phase, the probes may hybridize to their respective target sequences, and upon cleavage by the DNA polymerase, the fluorescent signals may be released and detected.

The sample may comprise a biological sample obtained from a subject. The biological sample may comprise serum, plasma, or other body fluids. In some cases, the biological sample may be obtained from a patient with HBV mono-infection, HDV mono-infection, or HBV-HDV co-infection. The method may be applied to clinical samples for diagnosing patients and monitoring viral suppression under antiviral therapy.

Prior to performing the one-step RT-qPCR, DNA and RNA may be purified from the sample. In some cases, the purified DNA and RNA may be eluted in a volume of approximately L. The purified nucleic acids may then be combined with the primer pairs, probes, and reaction mixture components.

The detection of the first fluorescent signal and the second fluorescent signal may be performed using a quantitative PCR instrument configured to detect the first fluorescent label in a first detection channel and the second fluorescent label in a second detection channel. The quantitative PCR instrument may capture fluorescence signals from both the FAM channel for HBV DNA quantification and the TAMRA channel for HDV RNA quantification. In some cases, a passive reference channel such as a ROX channel may be used as a passive control.

The method may exhibit robust target specificity with no carryover or contaminated fluorescence signal between the detection channels, even at high viral loads. The sensitivity for both HBV and HDV may be approximately 10 copies per microliter without significant interference between the two detection targets. The method may provide excellent linear amplification across a range of input template concentrations, enabling accurate quantification of viral loads across a wide dynamic range.

### [Kit for Simultaneous Detection and Quantification]

A kit for simultaneous detection and quantification of hepatitis B virus (HBV) DNA and hepatitis delta virus (HDV) RNA may comprise an HBV primer pair, an HDV primer pair, an HBV probe, and an HDV probe. The kit may provide all components for simultaneous quantification of HBV DNA and HDV RNA, enabling a single quantification process to obtain two readout reports for monitoring both viral loads.

The HBV primer pair may comprise an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2]. The HBV primers may be designed in genomic regions that are highly conserved among different HBV genotypes, as disclosed herein.

The HDV primer pair may comprise an HDV forward primer having an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4]. The HDV primers may be designed in conserved regions of the HDV genome. In some cases, the HDV forward primer may include a degenerate base Y to accommodate a CIT polymorphism between genotype 4 and other genotypes, enabling pan-genotypic detection of HDV.

The HBV probe may comprise a first fluorescent label attached to a 5′ end and a first quencher attached to a 3′ end. The HDV probe may comprise a second fluorescent label attached to a 5′ end and a second quencher attached to a 3′ end. The first fluorescent label and the second fluorescent label may be spectrally distinguishable, enabling simultaneous detection of both viral targets without interference between the two detection channels.

In some cases, the first fluorescent label may be carboxyfluorescein (FAM) and the second fluorescent label may be carboxytetramethylrhodamine (TAMRA). The FAM label may be used for HBV DNA detection and the TAMRA label may be used for HDV RNA detection. The FAM and TAMRA fluorophores may emit fluorescence at different wavelengths, providing excellent readout specificity with no cross-channel interference even at high viral loads.

In some cases, the HBV probe may comprise the nucleotide sequence AGATG+ATTA+GGCAGA+GGTGAAAAAG [SEQ ID NO. 5], wherein+denotes a locked nucleic acid modification. The locked nucleic acid modifications in the HBV probe may impart heightened structural stability and may increase hybridization melt temperature (Tₘ), enabling reliable detection even with shorter sequences in limited conserved regions.

In some cases, the HDV probe may comprise the nucleotide sequence GGTCC+G+ACCTGGGC+ATCCG [SEQ ID NO. 6], wherein+denotes a locked nucleic acid modification. The locked nucleic acid modifications in the HDV probe may enhance structural stability and may increase hybridization melt temperature for improved detection performance across diverse sample conditions.

The first quencher and the second quencher may be the same or different. In some cases, the first quencher and the second quencher may both quench across only a portion of the visible spectrum. The quenchers may be positioned at the 3′ end of the respective probes to suppress fluorescence emission until the probes are cleaved during amplification.

The kit may further comprise a reverse transcriptase and a DNA polymerase. In some cases, the kit may comprise a Warm Start-activated reverse transcriptase and a Hot Start DNA-dependent DNA polymerase. Including these enzymes enables a complete one-step RT-qPCR workflow for convenient and efficient viral quantification. The kit may further comprise dNTPs and buffer components combined into a single reaction mixture.

In some cases, the kit may comprise the primers at a final concentration of approximately 0.4 μM and the probes at a final concentration of approximately 0.2 μM in the single reaction system for HBV and HDV, respectively. The kit may be configured for use with a quantitative PCR instrument capable of detecting the first fluorescent label in a first detection channel and the second fluorescent label in a second detection channel.

The kit may be used to perform the method for simultaneous detection and quantification of HBV DNA and HDV RNA as disclosed herein. The kit may enable one-step RT-qPCR performed in a single reaction tube, reducing time and costs while simplifying the workflow for clinical and research applications. The kit may provide pan-genotypic detection capability across diverse HBV and HDV genotypes by targeting conserved regions in the respective viral genomes.

### [Dual Fluorescence Channel Detection System]

A dual fluorescence channel detection system for pan-genotypic simultaneous quantification of hepatitis B virus (HBV) DNA and hepatitis delta virus (HDV) RNA may comprise a reaction mixture and a quantitative PCR instrument. The dual fluorescence channel detection system may enable detection of all genotypes of genetically diverging HBV and HDV by targeting conserved regions in the respective viral genomes, providing a universal detection method for identifying different genotypes.

The reaction mixture may comprise an HBV primer pair targeting a conserved region of the HBV genome. The HBV primer pair may comprise an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2]. The HBV primer pair may target a highly conserved region of the HBV genome spanning nucleotides 1779-1883 across all HBV genotypes A through H. Through phylogenetic analysis and sequence alignment of representative HBV genome sequences spanning genotypes A through H, conserved regions may be identified for primer and probe design. In some cases, genetic proximity may be observed between genotypes A through E, while genotypes F through H may exhibit greater genetic distance.

The reaction mixture may further comprise an HDV primer pair targeting a conserved region of the HDV genome. The HDV primer pair may comprise an HDV forward primer having an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4]. The HDV primer pair may target a conserved region of the HDV genome spanning nucleotides 794-998 across all HDV genotypes 1 through 8. The HDV genome may exhibit substantial variation among different genotypes, with genetically close genotypes displaying approximately 83.79% genome identity and some genotypes displaying only approximately 70% identity compared to other genotypes. In some cases, the HDV forward primer may include a degenerate base Y to accommodate a C/T polymorphism between genotype 4 and other genotypes, enabling pan-genotypic detection of HDV.

The reaction mixture may further comprise an HBV probe comprising a first fluorescent label and a first quencher. The first fluorescent label may be attached to a 5′ end of the HBV probe and the first quencher may be attached to a 3′ end of the HBV probe. In some cases, the HBV probe may comprise the nucleotide sequence AGATG+ATTA+GGCAGA+GGTGAAAAAG [SEQ ID NO. 5], wherein+denotes a locked nucleic acid modification. The locked nucleic acid modifications may impart heightened structural stability and may increase hybridization melt temperature (Tₘ), enabling reliable detection even with shorter sequences in limited conserved regions.

The reaction mixture may further comprise an HDV probe comprising a second fluorescent label and a second quencher. The second fluorescent label may be attached to a 5′ end of the HDV probe and the second quencher may be attached to a 3′ end of the HDV probe. In some cases, the HDV probe may comprise the nucleotide sequence GGTCC+G+ACCTGGGC+ATCCG [SEQ ID NO. 6], wherein+denotes a locked nucleic acid modification. The locked nucleic acid modifications in the HDV probe may enhance structural stability and may increase hybridization melt temperature for improved detection performance.

In some cases, the first fluorescent label may be carboxyfluorescein (FAM) and the second fluorescent label may be carboxytetramethylrhodamine (TAMRA). The FAM channel may be used for HBV DNA detection and the TAMRA channel may be used for HDV RNA detection. The first fluorescent label and the second fluorescent label may emit fluorescence at different wavelengths, enabling simultaneous detection of both viral targets without interference between the two detection channels.

The dual fluorescence channel detection system may further comprise a quantitative PCR instrument configured to detect the first fluorescent label in a first detection channel and the second fluorescent label in a second detection channel. The quantitative PCR instrument may capture fluorescence signals from both the FAM channel for HBV DNA quantification and the TAMRA channel for HDV RNA quantification. In some cases, a passive reference channel such as a ROX channel may be used as a passive control. The dual fluorescence channel detection system may exhibit robust target specificity with no carryover or contaminated fluorescence signal between the detection channels, even at high viral loads.

In some cases, the HBV primer pair may be configured to specifically detect HBV relaxed circular DNA (rcDNA) through over-gap amplification. HBV pre-genomic RNA (pgRNA) and partially reverse-transcribed HBV single negative-strand DNA may be encapsidated and released into circulation in HBV patients. The over-gap amplification design may selectively target infectious HBV particles with rcDNA genomes while excluding non-infectious HBV particles that possess only short minus-strand DNA. During HBV replication, after initiation of encapsidation and reverse transcription, the polymerase conjugated primer may translocate from the 5′ prime DR1 to the 3′ prime DR1 to start reverse transcription, and the corresponding pgRNA template may be degraded by the RNase H domain of the polymerase. The degradation of the pgRNA template may abrogate PCR amplification for pgRNA in the one-step quantification. After reverse transcription of the HBV minus strand, the residual RNA primer may undergo a second template switch from 5′ DR1 to DR2 to initiate plus strand synthesis of HBV rcDNA, on which the primers may anneal and PCR may be performed. Based on the locations of the HBV primers, the reverse primer may have two binding sites on full-length pgRNA. The 3′ terminus of encapsidated and secreted pgRNA may be truncated, thereby eliminating one of the binding sites. The orientation of the paired primers, outward to each other, may avoid PCR amplification from pgRNA-derived cDNA templates, thereby exclusively amplifying and detecting HBV rcDNA. The over-gap amplification design may provide accurate quantification of viral load, particularly in samples from patients undergoing nucleos(t)ide analog therapy.

The reaction mixture may further comprise a reverse transcriptase and a DNA polymerase, as disclosed herein. In some cases, the reaction mixture may comprise a Warm Start-activated reverse transcriptase and a Hot Start DNA-dependent DNA polymerase. The reaction mixture may further comprise dNTPs and buffer components combined into a single reaction. The primers may be present at a final concentration of approximately 0.4 μM and the probes may be present at a final concentration of approximately 0.2 μM in the single reaction system for HBV and HDV, respectively.

The dual fluorescence channel detection system may provide pan-genotypic detection capability across diverse HBV and HDV genotypes. The sensitivity for both HBV and HDV may be approximately 10 copies per microliter without significant interference between the two detection targets. The dual fluorescence channel detection system may display linear amplification across a 7-log range of input template concentrations. The dual fluorescence channel detection system may provide reliable detection for HBV and HDV in vitro, in human liver chimeric mice, and in patient samples.

### Example

### Materials and Methods

Cell lines. 293 T (American Tissue Culture Collection, ATCC® Number: CRL-3216™, Manassas, VA) and HepG2 cell lines (American Tissue Culture Collection, ATCC © Number: HB-8065™, Manassas, VA) were maintained in Dulbecco's Modified Eagle Medium (DMEM; ThermoFisher, Waltham, MA) base medium supplemented with 10% (vol/vol) fetal bovine serum (FBS) (Sigma Aldrich, St Louis, MO). 293 T cells were grown on tissue culture-treated plastic ware (Corning Inc., Corning, NY) and HepG2 cells on type IV collagen-coated plates (Sigma Aldrich, St Louis, MO).

HBV and HDV dual channel one-step quantitative PCR. HBV and HDV qPCR primers were designed in genomic regions that are highly conserved among different genotypes obtained from Azenta Life Sciences (Burlington, MA). Both HBV and HDV probes were designed based on AFFINITY PLUS™ qPCR probe technology to impart heightened structural stability, leading to increased hybridization melt temperature (Tₘ) and were obtained from Integrated DNA Technologies (Coralville, IA). The primer and probe sequences and their 5′ fluorophore reporter dyes, 3′ quencher are listed in Table 1, below. For the One-Step quantitative PCR, the LUNA® Universal Probe One-Step RT-qPCR kit was used at a final concentration of 0.4 μM primers and 0.2 μM probes in the single reaction system for HBV and HDV, respectively. The PCR was performed at 55 °C for 10 mins, 95 °C for 1 min followed by 45-50 cycles at 95 °C for 15 s, and 61 °C for 40 s for signal capture.

TABLE 1
Primer and probe sequences used in this example

| Primer/Probe | 5′ to 3′ |
|---|---|
| HBV-qPCR-F | GGAGGCTGTAGGCATAAATTGG |
| | [SEQ ID NO. 1] |
| HBV-qPCR-R | CACAGCTTGGAGGCTTGAAC |
| | [SEQ ID NO. 2] |
| HBV-probe | FAM-AGATG+ATTA+GGCAGA+GGTGAAAAAG |
| | [SEQ ID NO. 5]-IB FQ |
| HDV-qPCR-F | GCGCCGGCYGGGCAAC |
| | [SEQ ID NO. 3] |
| HDV-qPCR-R | TCCTCTTCGGGTCGGCATG |
| | [SEQ ID NO. 4] |
| HDV-probe | TAMRA-GGTCC+G+ACCTGGGC+ATCCG |
| | [SEQ ID NO. 6]-IB RQSp |

Correlation and regression assays. The correlation and regression analysis of the quantifications between HBV-HDV dual and the HBV or HDV single detection system were performed using GraphPad Prism (Version 10.1.1, Boston, MA) software. Simple linear regression was used and goodness of fit was characterized by the R squared values from the Pearson r. 95% confidence intervals shown as dashed lines on the graphs. Two-tailed p values are shown to indicate the significance (alpha=0.05) of the correlation.

HBV and HDV transfection of human hepatoma cells. All work with infectious HBV and HDV has been reviewed and approved by the Princeton University Institutional Biosafety Committee (#1146). HepG2 cells were used for all transfection assays. Briefly, 1.2 E+5 or 7.0 E+4 cells/well were seeded in collagen-coated 24-well or 48-well plates, respectively. Within 24 h of seeding plates, cells were transfected with HBV-, HDV-encoding or empty backbone plasmids using OPTI-MEM™ Reduced Serum Medium (Thermo Fisher Scientific, Waltham, MA) and X-TREMEGENE™ HP DNA Transfection Reagents (St. Louis, MO). Total amount of transfected DNA was set to 0.5 μg/well for 24-well plates and 0.3 μg/well for 48-well plates. Six hours post transfection, the cells were washed at least 4 times. Supernatants were harvested on day 3 post transfection.

HDV full-length RNA in vitro transcription. HDV full-length DNA template was PCR amplified through the pSVL(D3) plasmid by HDV-T7-F and HDV-R. Then, the full-length 1.0×HDV DNA was purified and inserted into a TOPO-Blunt Cloning vector (Thermo Fisher, Waltham, MA) and the correct clone was then picked and plasmid DNA prepared using a miniprep kit (MACHEREY-NAGEL, Allentown, PA). The in vitro transcription template was then PCR amplified using the constructed vector with the same primers above and the full-length 1.0×HDV RNA was in vitro transcripted using a HiScribe T7 High Yield RNA Synthesis Kit (New England Biolabs, Ipswich, MA) following manufacturer's instructions. To remove template DNA, 70 μl nuclease-free water was added to the reaction system and then digested with DNase I (New England Biolabs, Ipswich, MA) for 15 min at 37 °C The RNA was then cleaned up through the MEGAclear kit (Thermo Fisher Scientific, Waltham, MA) following manufacturer's instructions. The purified HDV RNA was confirmed by running an RNA gel and the concentration was measured using a NanoDrop spectrophotometer (Thermo Fisher Scientific, Waltham, MA) and the copy number was calculated based on its molecular weight.

HBV minicircle standard preparation. The HBVcircle was prepared by using the parental construct in combination with the minicircle E. coli strain which has been specifically engineered to express the φ31 integrase and Scel endonuclease upon arabinose induction. Briefly, the HBV minicircle parental plasmid transformed colony was precultured for about 6 h in LB medium containing 50 μg/mL Kanamycin and then was transferred and cultured overnight in 400 mL TB medium with 50 μg/mL Kanamycin. When the OD600 of the overnight culture is between 4 and 5, a minicircle induction mix composed of 400 mL LB broth, 16 mL 1 N NaOH and 400 μL20% L-arabinose was combined with the bacteria medium and was incubated at 30 °C with shaking at 250 rpm for another 5 h or longer. Bacteria were pelleted and the HBVcircle DNA was extracted and purified using a NucleoBond Xtra Midi kit (MACHEREY-NAGEL, Allentown, PA).

To confirm the HBVcircle, the DNA with or without EcoR I digestion was run on a gel to see the supercoiled HBVcircle and the linearized 1.0×HBV genome. The HBVcircle was further characterized by transfecting into HepG2 cells to see the HBsAg production and HBV DNA levels in the supernatants.

Phylogenetic analysis of HBV and HDV genome variety among genotypes. The HBV DNA and HDV RNA sequences of different genotypes were retrieved from the National Center for Biotechnology Information (NCBI) and three representative genomes were used for each genotype except for HDV genotypes 1 and 6 for which an insufficient number of genome sequences were available. The phylogenic trees were built by MegAlign Pro (DNASTAR Inc., Madison, WI) using a RAXML alignment of HBV DNA or HDV RNA sequences among genotypes, respectively. The nucleotide numbering is based on the genome in the first line and different nucleotides are coloured.

Quantification of HBsAg and HBeAg. Detection and quantification of HBsAg and HBeAg levels were performed by a chemiluminescence immunoassay (CLIA) according to the manufacturer's instructions (Autobio Diagnostics CO., LTD, Zhengzhou, Henan, China) and the chemiluminescence was determined by a TriStar Multimode Reader (Berthold Technologies, Bad Wildbad, Germany).

DNA and RNA isolation from cell supernatants and mouse sera. The DNA and RNA were isolated using the QIAamp MinElute Virus Spin Kit (Qiagen, Hilden, Germany), following the manufacturer's instructions. Briefly, 20 μL were processed per sample and the purified DNA and RNA was eluted in 20 μL. To prepare supernatants for qPCR after infectious clone transfection, possible plasmid contamination was removed by DNase I (Thermo Fisher Scientific, Waltham, MA) treatment (37 °C for 1 h) prior to purification. Samples were then stored at −80 °C until further use.

Western blotting. Cells which had been seeded in a 24-well plate were lysed with 150 μL/well of 1×NuPAGE LDS buffer (Life Technologies, Carlsbad, CA) containing 2.5% (vol/vol) 2-mercaptoethanol (f-ME) at room temperature for 10 mins. The cell lysate was then transferred to a sterile 1.5 ml tube and heat-inactivated at 95 °C for 20 min. The samples were then loaded and separated by 10% (w/vol) sodium dodecyl sulfate-polyacrylamide gel electrophoresis (SDS-PAGE), and transferred to a nitrocellulose membrane (Roche, Basel, Switzerland). The membrane was blocked with 5% (w/vol) skim milk in Tris-buffered saline containing 0.1% Tween-20 (TBST) for 1 h at room temperature and then incubated overnight at 4 °C with the HBV core protein primary antibody (1:1000 dilution, rabbit polyclonal antibody) or HDAg antibody (1:1000 dilution, rabbit polyclonal antibody, a gift from John Taylor, Fox Chase Cancer Center, Philadelphia, PA). Next, the membranes were washed three times with 1× (Tris-Buffered Saline, 0.1% Tween® 20 Detergent) TBST and incubated with a goat anti-Rabbit IgG (1:5000 dilution; Thermo Fisher Scientific, Waltham, MA) secondary antibody at room temperature for 1 h. After further washing with TBST, the signal intensities of the membranes were scanned and visualized using an Odyssey CLx imager (Li-COR Biosciences, Lincoln, NE). The signal quantification was performed by ImageJ.

Generation of cell-culture derived HBV and HDV stocks by transfection. For HBV stocks, HepG2 cells were seeded into two 150—mm collagen-coated plates at 7.0 E+6 per plate. When cell confluence reached approximately 90%, the medium was replaced with 25 mL DMEM/F12 (Life Technologies, Carlsbad, CA), supplemented with 5% (vol/vol) FBS, 1% (vol/vol) penicillin/streptomycin (P/S) prior to DNA transfection. Cells in each plate were transfected with 28 μg plasmid DNA mixed with 2800 μL OPTI-MEM™ Reduced Serum Medium (#51985034, Thermo Fisher Scientific, Waltham, MA) and 84 μL X-TREMEGENE™ HP DNA Transfection Reagent (St. Louis, MO). After 4-6 h, cells were washed three times with 25-30 mL pre-warmed Dulbecco's Phosphate Buffered Saline (DPBS) and then cultured in 20 mL DMEM/F12 supplemented with 5% (vol/vol) FBS, 1% (vol/vol) P/S. After 2 h, cells were again washed twice with prewarmed 25-30 mL DPBS. Cells were subsequently cultured in 20 mL DMEM/F12 supplemented with 5% (vol/vol) FBS, 1% (w/vol) P/S. Supernatants were harvested every 2-3 days for 10-11 days and stored at 4 °C. Supernatants were centrifuged at 3000 g for 15 min at 4 °C to remove cell debris. Thereafter, supernatants were transferred into clean tubes and concentrated using heparin columns (GE Healthcare, Chicago, IL). Briefly, supernatants were run through the 5 mL heparin columns at a speed of 5 mL/min. Then the columns were washed once with 8 mL1×PBS followed by elution with 30 mL elution buffer (25 mL10×PBSpH 7.4 diluted into 100 mL with 75 mL distilled water). The elution was collected and further concentrated with the centrifugal filters (100,000 NMWL, Merck Millipore Ltd.) at 3000 g for 30 min at 4 °C. Finally, about 1.6 mL virus stocks were transferred and aliquoted in LoBind Microcentrifuge Tubes (Eppendorf, Hamburg, Germany). Then 20 μL of virus stock was used and DNase I (Thermo Fisher Scientific, Waltham, MA) was added (37 °C, 1 h) to digest possible residual plasmid DNA. DNA was extracted with the QIAmp MinElute virus spin kit (Qiagen, Hilden, Germany). Thereafter, HBV titres were quantified by qPCR as described here. Based on the HBV DNA copy numbers virus was aliquoted and cryopreserved at −80 °C until use. For HDV stocks, the production process was the same with HBV exception that 14 μg of each the HDV pSVL(D3) and HBV preS1 overexpression plasmids were transfected.

Generation of HepG2-hNTCP infection cell model. Lentiviral hNTCP pseudo-particles were generated as previously described. Briefly, 4.0 E+6 293 T cells were seeded in a 10 cm tissue-culture plate and plasmids expressing the respective pLVX-hNTCPtagRFP proviral DNA, HIV-1 gag-pol, and VSV-G at a ratio of 1/0.8/0.2 were co-transfected using Xtremegene (Sigma-Aldrich, St. Louis, MO). Supernatants were collected at 24, 48 and 72 hours post transfection, pooled, and filtered through a 0.45 μm filter (Millipore, Burlington, MA). Filtered lentiviral supernatants were supplemented with polybrene (MilliporeSigma, Darmstadt, Germany) (4 μg/mL, vol/vol) and (1:50, vol/vol) 1 M HEPES (Life Technologies, Carlsbad, CA), aliquoted, and stored at −80 °C until use. Next, HepG2 cells were transduced with the hNTCP-tagRFP encoding lentiviruses. After 3 days, expression of the fusion protein was assessed by both confocal microscope (Nikon A1R-STED, Nikon, Melville, NY) in the Princeton University Imaging Core facility and LSRII Multi-Laser Analyzer (BD, Franklin Lakes, NJ) at the Princeton University flow cytometry core facility. The flow cytometry data was analysed by FlowJo (Version 10.4, BD Biosciences, Franklin Lakes, NJ).

HBV and HDV infection of HepG2-NTCP cells. HepG2-NTCP cells were seeded into 48-well plates at 5.0 E+4 cells/well, and then were pre-treated with pretreat medium (DMEM supplemented with 3% (vol/vol) FBS, 2% (vol/vol) DMSO, 1% (vol/vol) Pen/Strep) for 24 h. HBV infections at a multiplicity of infection (MOI) of 8000 and HDV infections at a MOI of 1000 were proceeded in the presence of 4% polyethylene glycol (PEG) 8000 (Sigma-Aldrich, St. Louis, MO) and 2% dimethyl sulfoxide (DMSO, Sigma-Aldrich, St. Louis, MO). 12-24 h following inoculation, the cells were washed with pre-warmed sterile 1×PBS at least 4 times and then incubated with fresh maintenance media (DMEM supplemented with 3% FBS, 2% DMSO, 1% Pen/Strep and 1× nonessential amino acids). Samples were collected at the indicated time-points and stored at −80 °C until analysis.

In vivo experiments. All animal experiments were performed in accordance with protocols reviewed and approved by the Institution Animal Care and Use Committee (IACUC) of Princeton University (protocol number 3063).

The generation of Fah−/− NOD.Cg-Rag1^{tm1MomIL2rgtmlWjl/SzJ} IL2Rg^{null} (FNRG) mice has been previously described. Female FNRG mice older than 6 weeks of age were transplanted with ca. 1.0 E6 cryopreserved adult human hepatocytes. Primary hepatocytes were obtained from BioReclamation Inc. (Westbury, NY). FNRG mice were cycled on water supplemented with 2-(2-nitro-4-trifluoromethylbenzoyl)-1,3-cyclohexanedione (NTBC, Yecuris Inc., Tualatin, OR), to block the build-up metabolites to toxic concentrations. Approximately 3 months after transplantation, blood was collected by submandibular puncture and serum was separated by centrifuging the coagulated blood at 1100×g for 15 min. Mouse sera were analysed via human albumin ELISA, and mice with a serum concentration of >1000 μg/mL human albumin were chosen for the subsequent viral infections. For HBV mono- and for HBV-HDV super-infection, 3.0 E+8GE/animal HBV(200 μl volume with sterile 1×PBS) was inoculated by intraperitoneal (i.p) injection. In the HBV-HDV superinfection group, human chimeric mice were inoculated with 5.0 E+8GE/mouse HDV by i.p infection (200 μl volume diluted in sterile 1×PBS).

Human albumin ELISA for assessment of human hepatocyte engraftment of chimeric mice. Levels of human albumin in mouse serum were quantified by ELISA as described previously. Briefly, 96-well flat-bottomed plates (Nunc, Thermo Fisher Scientific, Waltham, MA) were coated with goat anti-human albumin antibody (1:500, #A80-129A, Thermo Fisher Scientific, Waltham, MA) in coating buffer (1.59 g Na₂CO₃, 2.93 gNaHCO₃, 1 LdH2O, pH=9.6) for 1 h at 37 °C. The plates were washed four times with wash buffer (0.05% (vol/vol) Tween 20 (Sigma Aldrich, St. Louis, MO) in 1×PBS), and then incubated with superblock buffer (Fisher Scientific, Hampton, NH) for 1 h at 37 °C. Plates were washed twice. Human serum albumin (Sigma Aldrich, St. Louis, MO) was diluted to 1 μg/mL in sample diluent (10% Superblock, 90% wash buffer), then serially diluted 1:2 in 135 μL sample diluent to establish an albumin standard. Mouse serum (5 μl) was used for a 1:10 serial dilution in 135 μL sample diluent. The coated plates were incubated for 1 h at 37 °C, then washed three times. Mouse anti-human albumin (50 μL, 1:2000 in sample diluent, Abcam, Cambridge, UK) was added and plates were incubated for 2 h at 37 °C. Plates were washed four times and 50 μL of goat anti-mouse-horse radish peroxidase (HRP, 1:10,000 in sample diluent, LifeTechnologies, Carlsbad, CA) were added and incubated for 1 h at 37 °C. Plates were washed six times. Tetramethylbenzidine (TMB, 100 μL) substrate (Sigma Aldrich, St. Louis, MO) was added and the reaction was stopped with 25 μL2—NH₂SO₄. Absorbance was read at 450λ on the BertholdTech TriStar (Bad Wildbad, Germany).

Quantification of HBV pgRNA by quantitative RTPCR. HBV RNA purified from supernatants was used to determine extracellular nucleocapsid-associated HBV pre-genome RNA (pgRNA) level as described previously. Briefly, 7.5 μl of isolated nucleic acid was treated with DNase I (Thermo Fisher Scientific, Waltham, MA) followed by reverse transcription with a specific HBV primer (5′-CGAGATTGA-GATCTTCTGCGAC-3′, nt 2415-2436, numbered based on gt D with GenBank accession no. U95551.1) located in precore/core region and anchored sequences using RevertAid™ First Strand DNA Synthesis Kit (Thermo Fisher Scientific, Waltham, MA, USA). For quantitative assays, the standards with 1-mer HBV target template were cloned into the TOPO-Blunt Cloning vector (Thermo Fisher, Waltham, MA, USA #450245) and the copy number was calculated based on the number of nucleotides of the plasmid and concentration of the plasmid preparation. A master mix was created containing 15 μl2×Taqman reaction mix (Applied Biosystems, Waltham, MA, USA), 500 nM forward and reverse primers, 200 nM probe and 3 μl synthesized cDNA in a 30 μL reaction. The master mix was then added to the samples or to the 10-fold serial dilution standards and the following cycling programme was used to run the qPCR: 95 °C for 10 min; 45 cycles at 95 °C for 15 sec and 58 °C for 45 sec.

Immunofluorescence of HBcAg and HDAg. For infection samples, HepG2 cells +/−NTCP were grown on collagen-precoated cover slides and fixed 6 days after infection with 4% (vol/vol) paraformaldehyde (PFA). For transfection samples, HepG2 cells were grown on collagen-precoated cover slides and fixed 3 days after transfection with 4% (vol/vol) PFA. The cells were then washed 3 times with 1×PBS followed by 1 h of incubation with blocking and permeabilization buffer (5% BSA, 5% FBS, 0.3% Tri-ton-X-100 in 1×PBS, all (vol/vol)) at room temperature. Thereafter, cells were incubated with either antiHBc antibody (1:500 dilution, #B0586, Dako, Denmark) or anti-HDAg (1:250 dilution, a gift from John Taylor, Fox Chase Cancer Center, Philadelphia, PA), respectively in the dilution buffer (1% BSA, 1% FBS, 0.3% Triton-X-100 all (vol/vol) in 1×PBS) at 4 °C overnight, washed for 3 times and incubated with an Alexa Fluor 488 conjugated goat anti-rabbit secondary antibody (1:1000 dilution, #A11008, Thermo Fisher Scientific, Waltham, MA) and DAPI (1 μg/mL) in dilution buffer (1% BSA, 1% FBS, 0.3% Triton-X-100 all (vol/vol) in 1×PBS) at room temperature for 1 h. After 3 washes, the cover slides with cells were then transferred onto a glass slide with 2 μl ProLong Gold antifade solution (#P36930, Thermo Fisher Scientific, Waltham, MA) and sealed with COVERGRIP™ Coverslip Sealant (#23005, Biotium Inc., Fremont, CA). The slides were then covered with foil and dried overnight at 4 °C and subsequently analysed with a confocal microscope (Nikon A1R-STED, Nikon, Melville, NY) in the Imaging Core facility at Princeton University.

Histology processing and Multiplexed HDAg, HBcAg and human 02-macroglobulin immunohistochemistry. Tissue samples were fixed for a minimum of 72 h in 4% (w/vol) paraformaldehyde (PFA) before processing in a Tissue-Tek VIP-5 automated vacuum infiltration processor (Sakura Finetek USA, Torrance, CA) and embedded in paraffin using a HistoCore Arcadia paraffin embedding machine (Leica, Wetzlar, Germany). 5-μm tissue sections were generated using a RM2255 rotary microtome (Leica) and transferred to positively charged slides, deparaffinized in xylene, and dehydrated in graded ethanol. A Ventana Discovery Ultra tissue autostainer (Roche, Basel, Switzerland) was used for multiplex fluorescent immunohistochemistry (mfIHC). Briefly, tyramide signal amplification (TSA) was used in an iterative approach to covalently bind Opal fluorophores (Akoya Bioscience, Marlborough, MA) to tyrosine residues in tissue. Sequential heat-stripping was performed on each primary-secondary antibody complex following each fluorophore application until all antibodies were developed. Liver tissue from non-humanized and non-infected FNRG mice was used as a negative control for the immunohistochemistry assay to confirm the specificity of antibodies. All primary antibodies were of rabbit origin, and thus developed with a secondary goat anti-rabbit HRP-polymer antibody (Vector Laboratories, Burlingame, CA) for 20 min at 37 °C. All Opal TSA-conjugated fluorophore reactions took place for 20 min. Slides were counterstained with spectral DAPI (Akoya Biosciences, Marlborough, MA) for 16 min before being mounted with ProLong Gold Antifade Mountant (ThermoFisher, Waltham, MA).

Multispectral microscopy. Fluorescently labelled slides were imaged using a Vectra Polaris™ Quantitative Pathology Imaging System (Akoya Biosciences, Marlborough, MA). Exposures for all Opal dyes were established utilizing regions of interest with strong signal intensities to minimize exposure times and maximize the specificity of the signal detected. To maximize signal-to-noise ratios, images were spectrally unmixed using a synthetic library specific for each Opal fluorophore and DAPI. Furthermore, an unstained section was used to create an autofluorescence signature that was subsequently removed from whole-slide images using InForm software version 2.4.8 (Akoya Biosciences, Marlborough, MA). InForm exported the multispectrally unmixed images as QPtiffs, which were then fused together as a single whole slide image in HALO (Indica Labs, Albuquerque, NM) for acquisition of representative images.

Serum samples from HBV and HBV/HDV positive patients. Serum samples from deidentified HBV+ patients were obtained from the American Red Cross, and samples from HBV+/HDV+ patients from ID Care. This work was deemed non-human subjects research by the Princeton University Institutional Review Board (IRB).

HBV genome cloning from patient serum. HBV DNA was isolated by processing 20 μL patient serum through the QIAamp MinElute Virus Spin Kit (Qiagen, Hilden, Germany). HBV full-length genomes were then PCR-amplified using Hi-fidelity polymerase (PRIMESTAR® GXL DNA Polymerase, Takara Bio USA, San Jose, CA) by HBV-F (5′-CCGGAAAGCTTGAGCTCTTCTTTTTCACCTCTGCCTAATCA [SEQ ID NO. 1]-3′) and HBV-R (5′-CCGGAAAGCTTGAGCTCTT-CAAAAAGTTGCATGGTGCTGG [SEQ ID NO. 2]-3′) which can anneal to regions that are conserved across the different HBV genotypes. The PCR product was then purified and then inserted into a TOPO-Blunt Cloning vector (Thermo Fisher, Waltham, MA) following by Sanger sequencing (Azenta Life Sciences, Burlington, MA).

Statistical Analysis. Bar graphs were presented as mean±SEM with individual data points and at least three biological independent replicates were performed. All data was analysed with Prism v9.2.0 (GraphPad) and Student's t test (unpaired, two-tailed) was used for the statistics. Statistical significance of any p values is indicated.

### Results

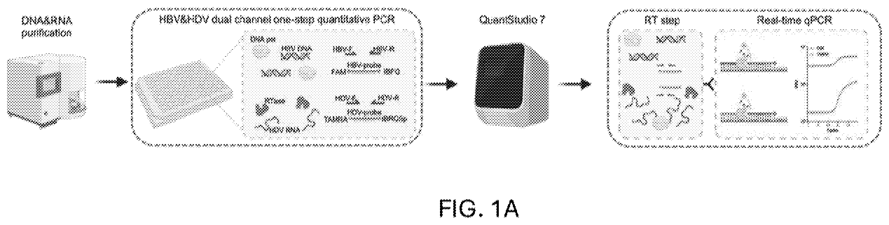

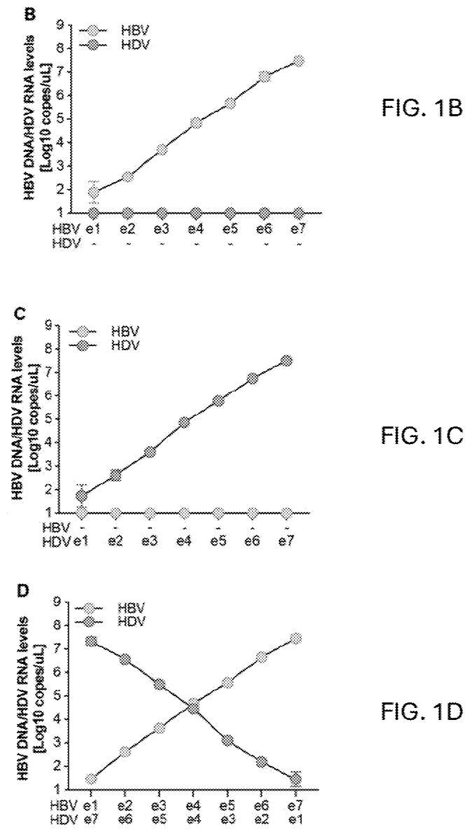

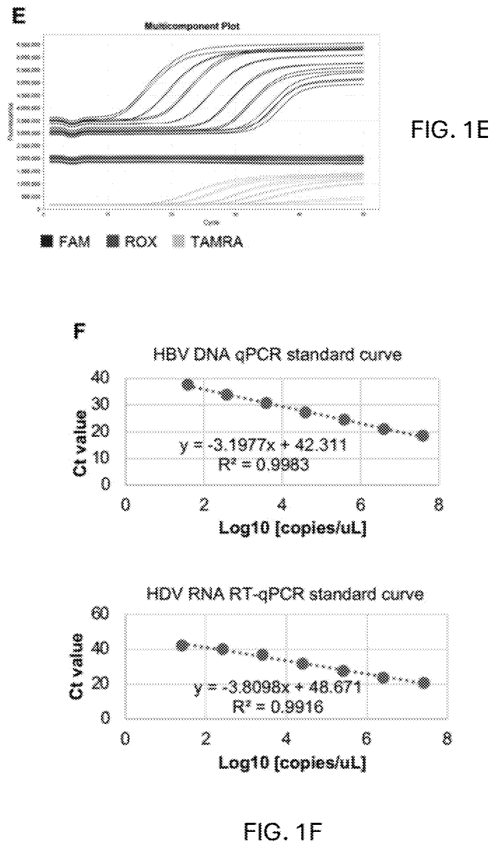

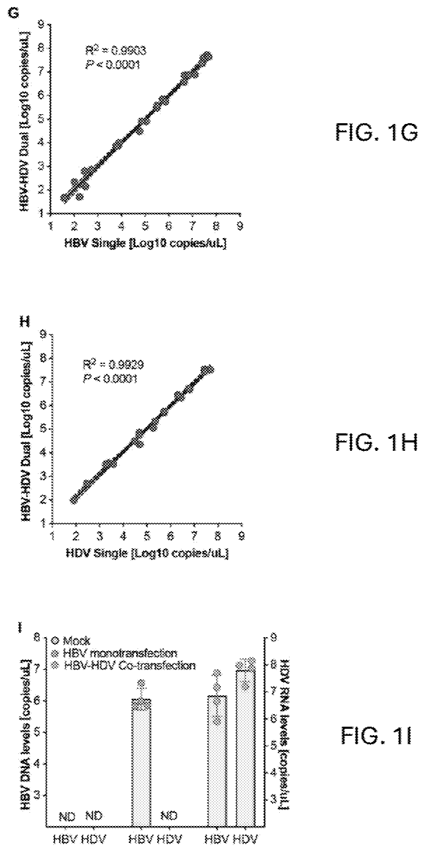

Characterization and validation of the sensitivity, specificity, and accuracy of the HBV-HDV dual channel quantitative detection system. The HBV-HDV dual channel quantitative detection system was developed by employing a one-step RT-qPCR system with Warm Start-activated reverse transcriptase, Hot Start DNA-dependent DNA polymerase, dNTPs, and all the necessary buffer components into a single reaction. For simultaneous quantification of HBV and HDV, specific FAM (Carboxyfluorescein) and TAMRA (Carboxytetramethylrhodamine) labelled probes were designed targeting the respective genomes (FIG. 1A). HDV quantification standards were prepared through in vitro transcription of full-length negative-strand HDV RNA to closely mimic the authentic HDV genome. Standards for HBV DNA were created using the HBV minicircle technology, generating 1×HBV genome molecules. The amplification efficiency of the HBV DNA qPCR and HDV RNA RT-qPCR systems was individually confirmed.

To assess the dual application of two fluorophores in a single reaction system, a serial dilution of HBV (ranging from 10-1 e7 copies) was used as the template, without input HDV. The results demonstrate that the HBV-HDV dual channel quantitative detection system exhibits robust target specificity, showing no carryover or contaminated fluorescence signal from the FAM channel to the TAMRA channel, even at the highest HBV levels (FIG. 1). This observation was validated running the assay on serially diluted HDV, demonstrating no carryover or contaminated fluorescence signal from the TAMRA channel to the FAM channel, even at the highest HDV levels (FIG. 1C).

Further experimentation, involving gradually increased HBV templates and simultaneously decreased HDV templates, demonstrated that the HBV-HDV dual channel quantitative detection system maintained excellent readout specificity (FIGS. 1D-1E) and sensitivity, displaying exceptional linear amplification across a 7-log range (1 e1 to 1 e 7) of input template concentrations (FIG. 1F). Correlations of quantifications between the HBV-HDV dual detection system and the HBV (FIG. 1G) or HDV (FIG. 1H) single detection systems indicated similar accuracy.

To validate its applicability, the HBV-HDV dual detection system was tested in a transfection culture model, demonstrating its ability to detect HBV DNA only in the supernatant of the HBV mono-transfection group. Moreover, the platform could capture and quantify both HBV DNA and HDV RNA simultaneously in the HBV-HDV co-transfection group (FIG. 1I).

The HBV and HDV dual channel system can be used for pan-genotype specific HBV DNA and HDV RNA detection. HBV exhibits remarkable genetic heterogeneity owing to its error-prone reverse transcriptase/polymerase, resulting in its phylogenetic classification into at least nine genotypes (A to I). Similarly, the genetic diversity of HDV is substantial, leading to the identification of eight major clades based on phylogenetic classification derived from their genome sequences. Although distinct genotypes of HBV or HDV may traditionally align with specific ethno-geographic regions, contemporary population migration and increased mobility have contributed to a more pan-genotypic prevalence globally. Consequently, the development of a universal, comprehensive pan-genotypic detection system would have great utility for monitoring HBV and HDV infections and quantifying viral loads across diverse patient populations.

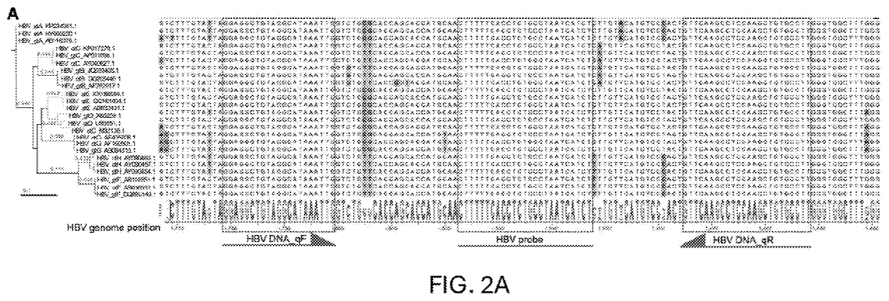

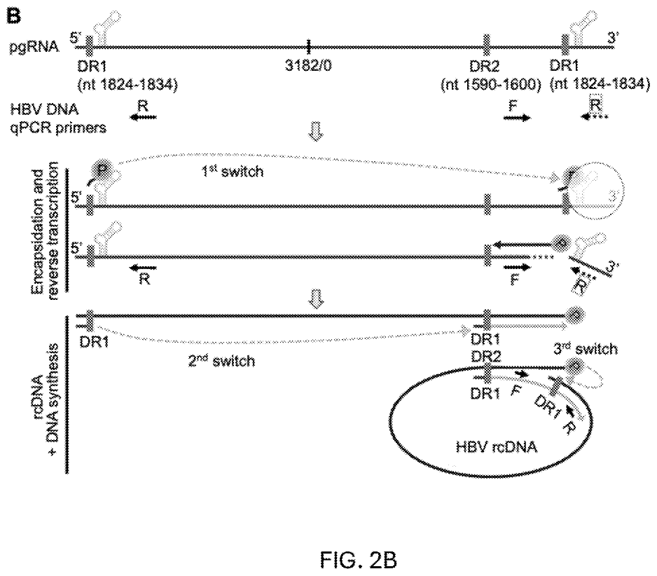

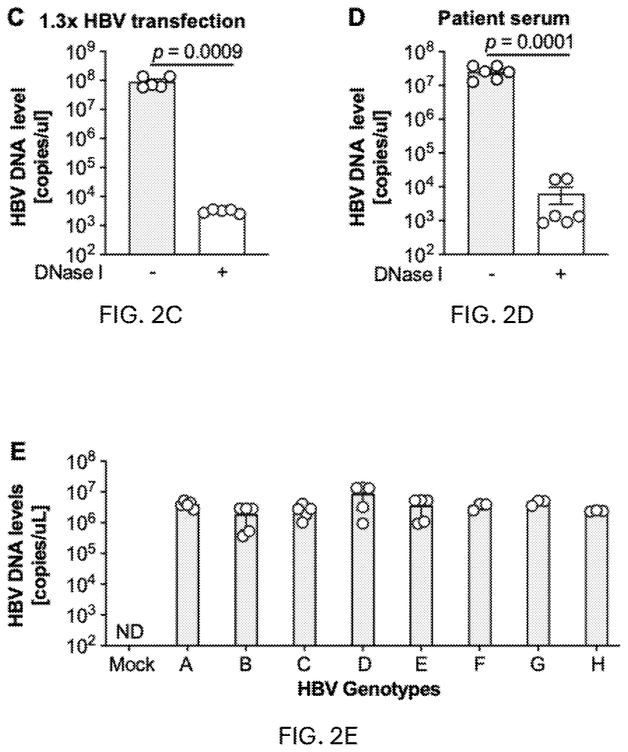

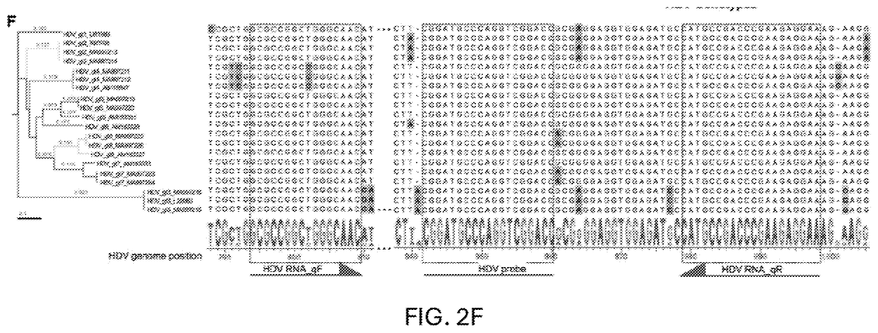

Through phylogenetic analysis and sequence alignment of representative HBV genome sequences spanning genotypes A to H, genetic proximity was observed between genotypes A-E, while genotypes FH exhibited greater genetic distance (FIG. 2A). Given that HBV pre-genomic RNA (pgRNA) and partially reverse-transcribed HBV single negative-strand DNA may be encapsidated and released into the circulation in HBV patients, primers and probes were designed for HBV quantification to specifically detect the authentic HBV relaxed circular DNA (rcDNA) genome in highly conserved regions (nt 1779-1883) across all genotypes (FIG. 2A). After the initiation of encapsidation and reverse transcription, the polymerase (TP domain) conjugated primer translocates from the 5′ prime DR1 to the 3′ prime DR1 to start the reverse transcription (1st switch) and at the same time the corresponding pgRNA template is degraded by the RNase H domain of the polymerase, which abrogates PCR amplification for pgRNA in the one-step quantification. After reverse transcription of the HBV minus strand, the residual RNA primer has a second template switch from 5′ DR1 to the DR2 to initiate the plus strand synthesis of HBV rcDNA, on which the primers can be annealed and the PCR can be performed (FIG. 2B). Based on the locations of the HBV primers, the reverse primer may have two binding sites on the full-length pgRNA (FIG. 2B). The 3′ terminus of encapsidated and secreted pgRNA is truncated, thus eliminating one of the binding sites. The orientation of paired primers, outward to each other, avoids PCR amplification from pgRNA-derived cDNA templates, thereby exclusively amplifying and detecting HBV rcDNA.

This was corroborated by using supernatants from cells transfected with a plasmid encoding a 1.3×HBV gt D genome and patient serum, where DNase I treatment resulted in a significant reduction of detected HBV DNA levels (FIGS. 2C-2D). The pan-genotype specificity of HBV DNA quantification was further validated using supernatants transfected with infectious clones from genotypes A to H, respectively (FIG. 2E).

Similarly, the HDV genome exhibits substantial variation among different genotypes, with genetically close genotypes 2 and 5 displaying only 83.79% genome identity (FIG. 2F). Genotype 3 of HDV stands out with only approximately 70% identity compared to the remaining seven genotypes. Primers and probes for detecting HDV RNA by RT-qPCR were carefully designed in conserved regions (nt 794-998) (FIG. 2F). Notably, despite a single T/C nucleotide polymorphism within the forward primer for genotype 5, previous studies have demonstrated the functionality of this primer for genotype 5 HDV quantification. Therefore, the HDV quantification system covers all known genotypes.

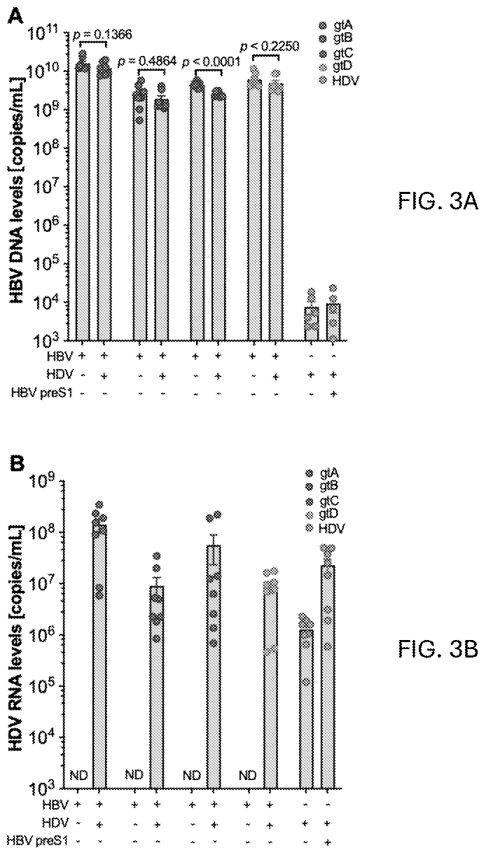

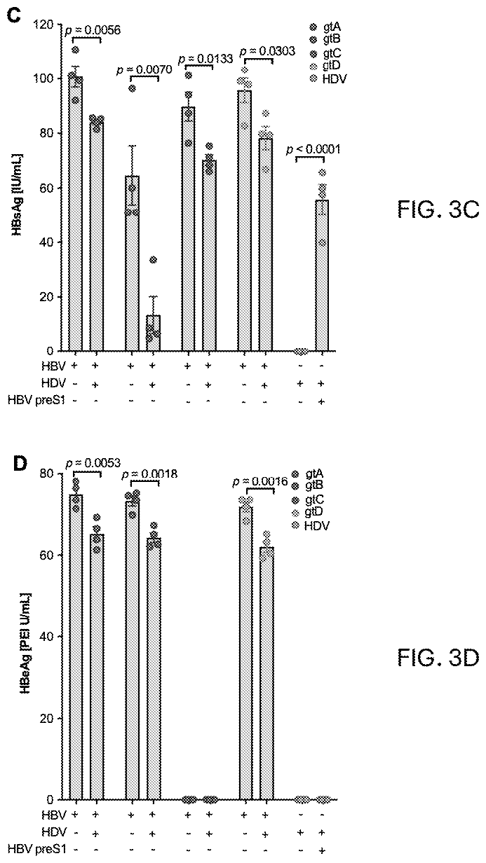

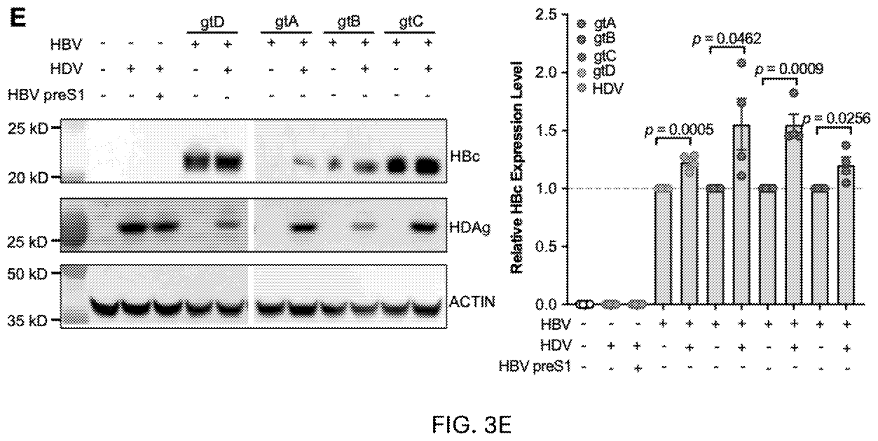

The HBV and HDV dual channel quantification system can be applied to investigate the HBV and HDV interactions. Patients with chronic hepatitis delta and B experience accelerated liver disease progression compared to those with HBV infection alone. Investigating the coexistence of HBV and HDV and their interaction within hepatocytes is crucial for understanding the underlying mechanisms of viral antigen expression, viral persistence, and gaining insights into the severe pathogenesis of HBV and HDV coinfection. Utilizing the infectious clones of HBV genotypes A to D developed previously and an HDV replication-competent plasmid pSVL(D3), both HBV mono-transfection and HBV and HDV co-transfection experiments were conducted on HepG2 cells. To study HDV alone, an HBV envelope protein expression vector was constructed to assemble and secrete HDV virions. The results demonstrate the specificity of the dual-channel quantification system in detecting HBV DNA and HDV RNA, respectively (FIGS. 3A-3B).

Compared with HBV mono-transfection, the levels of HBV DNA and HBV pre-genomic RNA (pgRNA) were lower when HDV was co-transfected, particularly notable for HBV genotype C (FIG. 3A) presumably because its replication-enhancing mutations. Moreover, the secretion of HBsAg and HBeAg was significantly reduced in the HBV and HDV co-transfected group (FIGS. 3C-3D), suggesting that HDV may inhibit HBV replication machinery or the secretion processes. Notably, no HBeAg was detected for HBV genotype C due to basic core promoter mutations in the clinically isolated genome strain. Western blot experiments demonstrated that, contrary to the decreased secretion of HBV viral markers, there was an accumulation of HBV core protein for all HBV genotypes in the HBV and HDV co-transfected groups (FIG. 3E). Intracellular hepatitis delta antigen (HDAg) levels, on the other hand, were decreased in the presence of HBV envelope protein or HBV infectious clones, which are essential for HDV assembly and secretion (FIG. 3E).

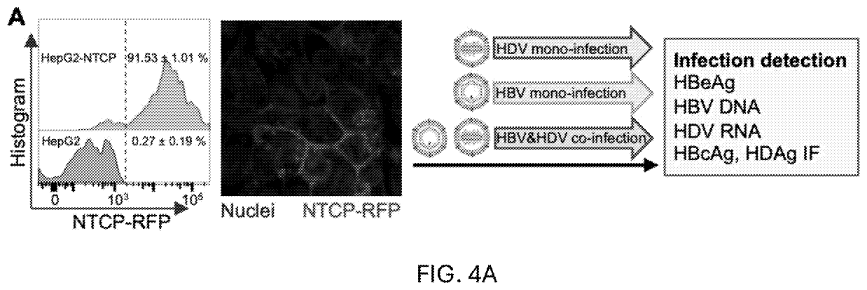

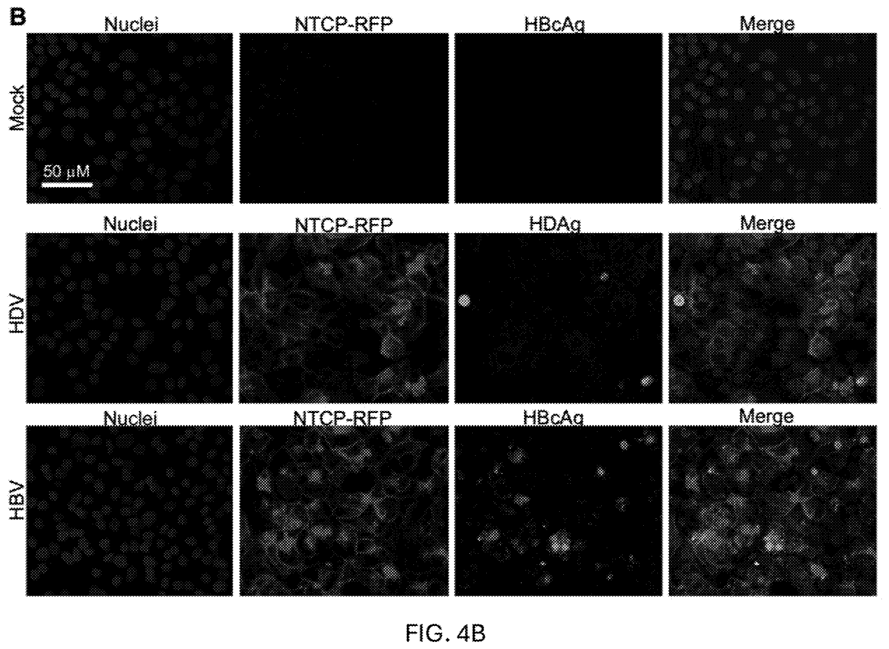

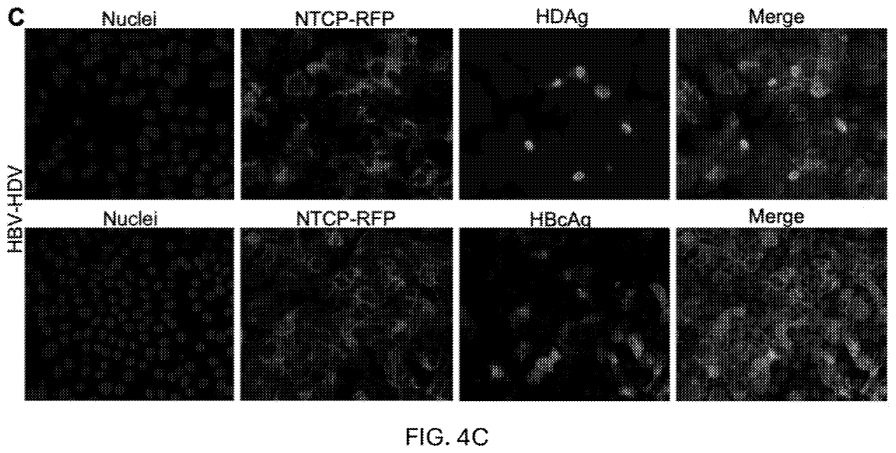

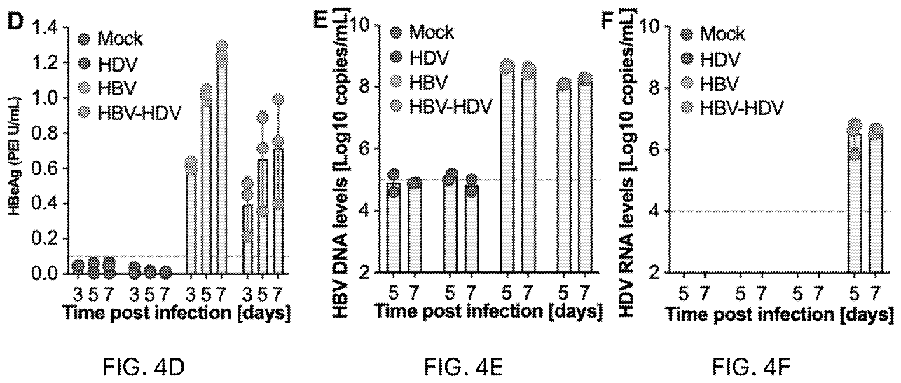

The HBV and HDV dual channel quantification system can be used to investigate HBV and HDV co-infection in vitro. Next, the previously established HepG2-NTCP in vitro infection system was employed to assess the functionality of the HBV and HDV dual channel quantification system. NTCPRFP expression and membrane localization were validated through flow cytometry and confocal imaging, after which the cells were utilized for HBV monoinfection, HDV mono-infection, and HBV-HDV coinfection experiments (FIG. 4A). To verify authentic infection, immunofluorescence assays targeting HBcAg and HDAg were conducted at 5 days postinfection, with the antibodies validated in the transfection system. Consistent with previous observations, HDV established infections in HepG2-NTCP cells (FIG. 4B, middle), while significant HBcAg-positive staining was observed in the HBV mono-infection group (FIG. 4B, bottom). In the HBV and HDV co-infection scenario, both HBcAg and HDAg were detected (FIG. 4C).

Successful HBV infection in both the HBV monoand HBV-HDV co-infection groups was further confirmed by the gradual increase in HBeAg levels in the supernatants (FIG. 4D). Subsequently, HBV and HDV dual channel quantification was performed using supernatants collected at days 5 and 7 post-infection. The results revealed that the dual channel quantification system can efficiently quantify HBV DNA and HDV RNA simultaneously in the HBVHDV co-infection group, whereas only HBV DNA was detected in the HBV mono-infection group (FIGS. 4E-4F). This underscores the robust applicability of the quantification system for in vitro infection assays, opening the possibility for concurrent monitoring of HBV and HDV dynamics during co-infection scenarios.

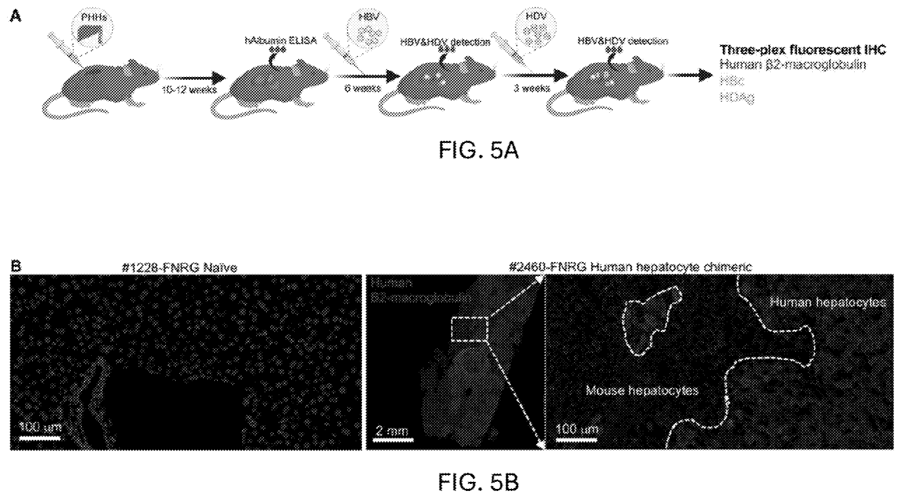

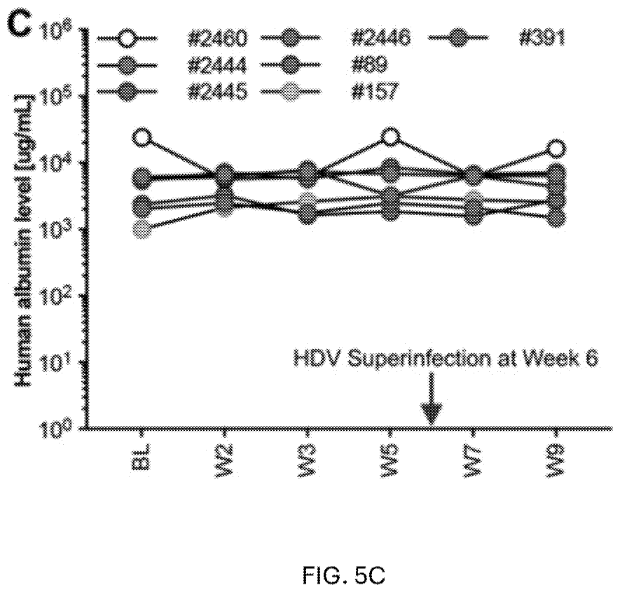

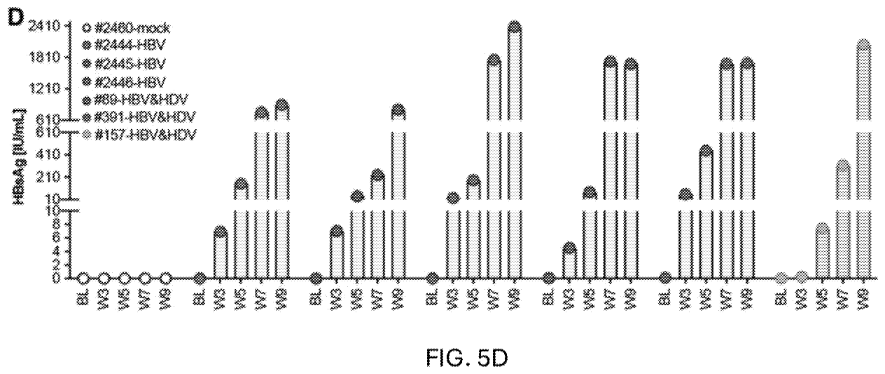

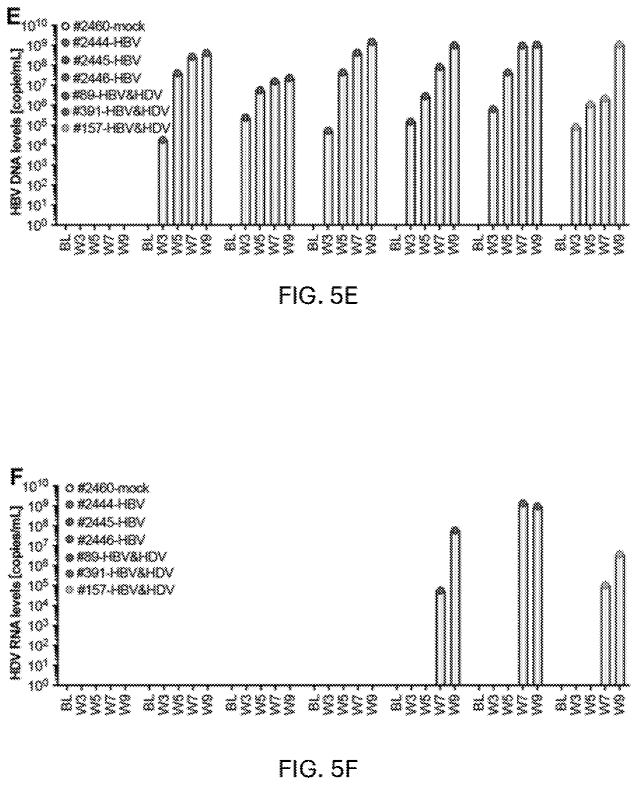

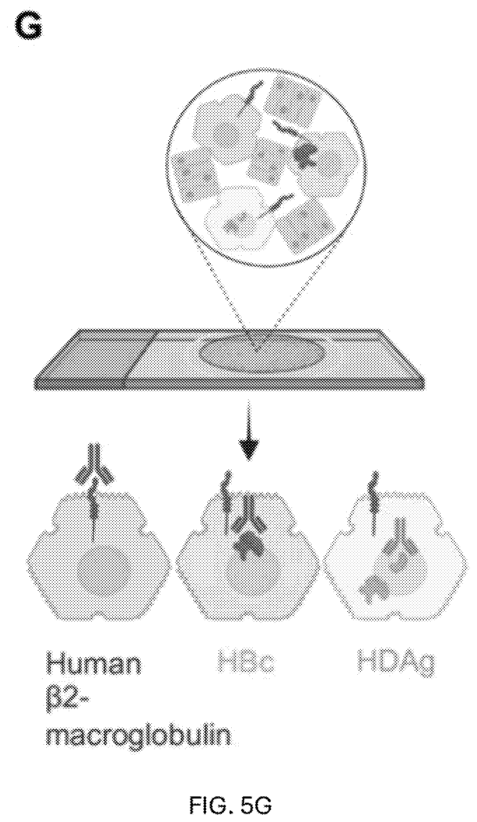

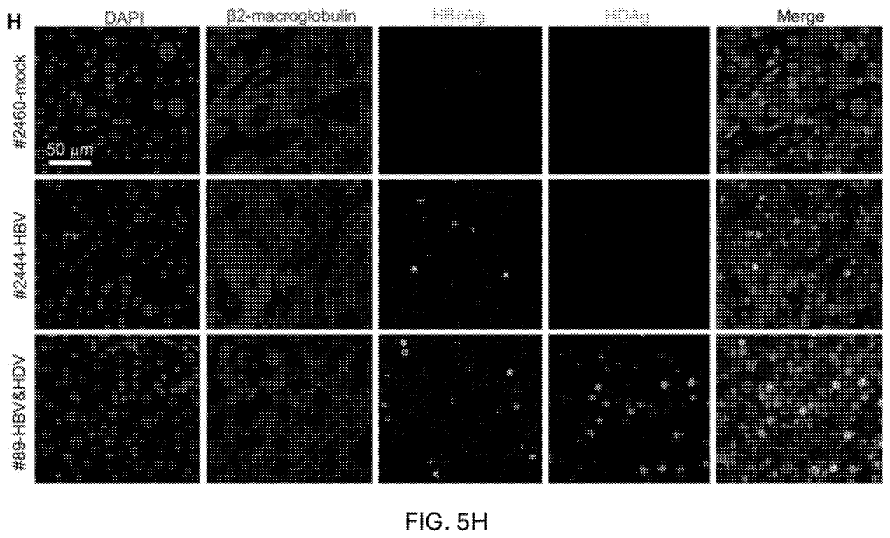

The HBV and HDV dual channel quantification system can be used to investigate HBV and HDV infection in vivo in human liver chimeric mice. To evaluate the performance of the HBV and HDV dual-channel quantification system in vivo, human liver chimeric FNRG mice were generated for HBV infection and HBV-HDV superinfection (FIG. 5A). Liver tissue from human liver chimeric mice was histologically characterized using human-specific β2-macroglobulin in situ staining (FIG. 5B) to visualize human hepatocyte engraftment. Mice with human albumin levels exceeding 1000 μg/mL at 10-12 weeks post-human hepatocyte transplantation were selected for mock infection (n=1), HBV mono-infection (n=3), and HBV-HDV superinfection (n=3). Human albumin levels were monitored longitudinally (FIG. 5C). Increases in serum HBsAg levels indicated successful HBV infection in both HBV mono- and HBV-HDV superinfection groups (FIG. 5D), with detectable circulating HBV DNA levels at 3 weeks post-infection (FIG. 5E).

At week 6 post-HBV infection, when the HBV loads exceeded 1 E+6 copies/mL in mouse serum, the mice in the HBV-HDV superinfection group were intraperitoneally inoculated with HDV (5 E+8 copies/animal). Utilizing the HBV and HDV dual channel quantification system, only HDV RNA was detected in the HBV-HDV superinfection group (FIG. 5F), indicating the suitability of the dual channel quantification system for accurate HBV and HDV detection in vivo.

Finally, multiplex fluorescent immunohistochemistry was employed to simultaneously detect human β2 macroglobulin, HBc, and HDAg on the same tissue section simultaneously (FIG. 5G). Human β2—macroglobulin staining confirmed robust human hepatocyte engraftment in mouse livers (FIG. 5H). Consistent with serological viral markers, specific HBcAg staining was observed in both HBV monoand HBV-HDV superinfection mice, while HDAg was detected exclusively in liver of the HBV-HDV superinfection groups (FIG. 5H). This comprehensive analysis reaffirms the successful establishment of the HBV-HDV superinfection model and highlights the dual-channel quantification system's efficacy for accurate detection and monitoring of HBV and HDV dynamics in an in vivo setting.

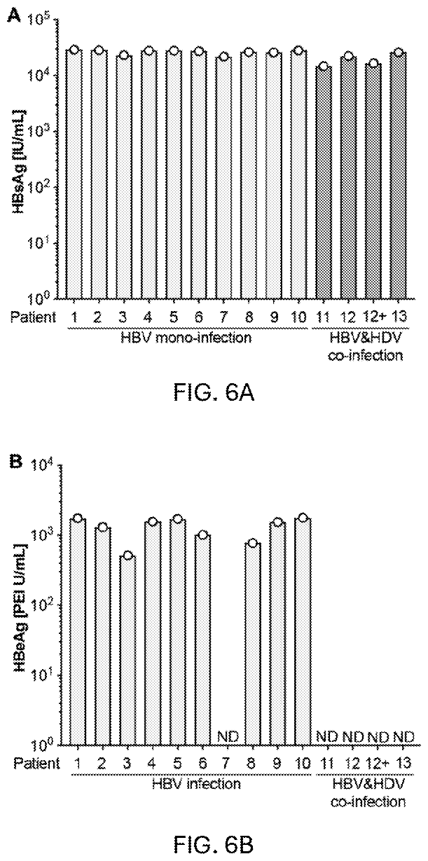

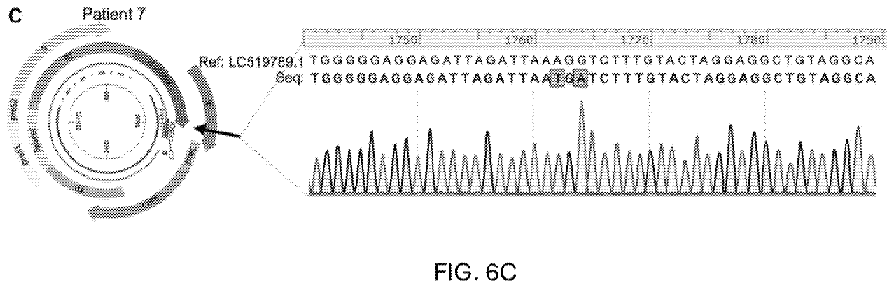

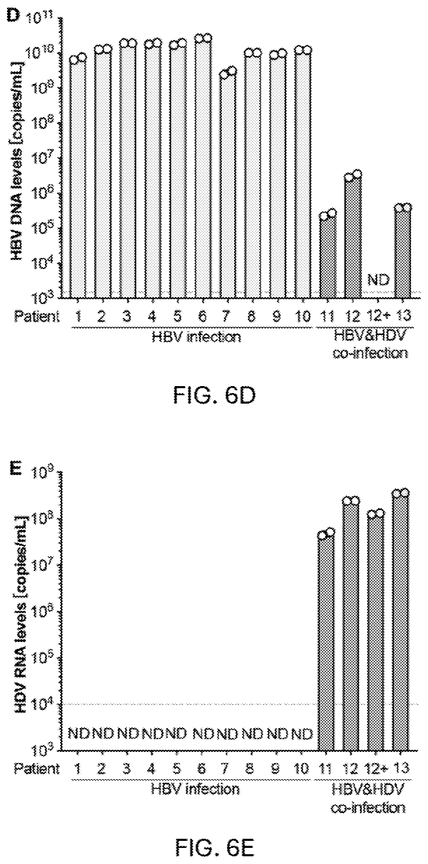

Preclinical assessment of the dual fluorescence channel HBV/HDV quantitative detection system on serum from patients with HBV mono-infection or HBV-HDV coinfection. The evaluation of the dual fluorescence channel HBV-HDV quantitative detection system was extended to patient samples, including those from patients with HBV mono-infection (n=10) and HBV-HDV coinfection (n=3). The HBV-HDV coinfected patients were undergoing antiviral therapy with the first-line treatment drug entecavir (ETV, patient 12) or tenofovir disoproxil fumarate (TDF, patient 13) for an unspecified duration. Patient 12 had two blood draws at month 6 and month 17 post ETV therapy. HBsAg tests indicated that all patients exhibited very high levels (>10,000 IU/mL) of HBsAg, and the nucleos(t)ide analogue (NA) treatment did not significantly reduce HBsAg levels (FIG. 6A). However, after ETV or TDF treatment, none of the HBV-HDV coinfection patients showed detectable HBeAg in the serum (FIG. 6B), aligning with clinical observations that prolonged nucleos (t) ide analogue therapy can lead to low levels or even undetectable HBV DNA and HBeAg seroconversion. Notably, patient 7 displayed undetectable HBeAg even without antiviral treatment.

To understand the molecular basis for the apparent absence of HBeAg expression in patient 7, the HBV genome from this patient was cloned. Sequencing results revealed A1762T and G1764A mutations in the core promoter (CP), basal core promoter (BCP), and Enhancer II overlapping gene expression regulatory regions and the G1896A mutation in the precure region (FIG. 6C). These regions have previously been identified as crucial for HBeAg production, and G1896A mutation here can abrogate HBeAg expression, while T1762A and A1764G back mutations can significantly restore HBeAg levels.

Using the dual fluorescence channel HBV-HDV quantitative detection system, high levels of HBV DNA were observed in all 10 patient samples without antiHBV treatment, while patients in the HBV-HDV coinfection group undergoing NA therapy exhibited much lower (>3 log) or undetectable (patient #12) HBV DNA levels (FIG. 6D). As expected, the dual-channel system selectively detected and quantified HDV RNA levels only in the HBV-HDV coinfection patients, not in the HBV mono-infection samples (FIG. 6E). This underscores the system's efficacy in accurately distinguishing and quantifying HBV and HDV in clinical samples, providing valuable insights into the virological dynamics of coinfection and the impact of antiviral therapy.

The prevalence of HDV is likely underestimated due to the limited application of reliable HDV RNA detection in HBV patients. HDV infection is closely associated with HBV infection, indicating that HDV can only coexist in individuals who are either HBV infected or at least HBsAg positive. Given the persistent challenge of achieving HBsAg clearance for HBV patients, they remain at a high risk of HDV superinfection throughout their lives. Therefore, monitoring HBV DNA and potential HDV RNA is crucial.

Notably, the co-infection of HBV and HDV can expedite the progression from chronic hepatitis to end-stage liver diseases, significantly increasing morbidity and mortality. Early diagnosis of HDV infection is paramount, and universal screening in HBV patients is both feasible and cost-effective. The disclosed HBV-HDV dual quantification system offers a timely and cost-efficient solution. Through a single quantification process, one can obtain two readout reports, allowing simultaneous monitoring of HBV and HDV levels.

Presently, HDV diagnosis heavily relies on blood tests for HDV antibodies (IgG and IgM), though this method fails to distinguish ongoing from past infections. Moreover, serological detections often lack sensitivity and quantification capabilities. The gold standard for diagnosing ongoing HDV infection is the presence of HDV RNA and viremia. Several RT-qPCR-based in-house HDV quantification methods have been developed, and more recently, a CRISPR/Cas13a-based HDV RNA detection technology with high sensitivity has been introduced.

However, existing assays typically focus on a single target, quantifying HDV RNA alone, thereby limiting the ability to monitor dynamic interactions between HBV and HDV. Furthermore, some assays employ HDV DNA plasmids or cDNA as standards for HDV RNA quantification, raising concerns about the calibration of reverse transcription efficiency. This inconsistency makes the reliability of HDV RNA quantification from different labs questionable and incomparable.

In this study, these issues were addressed by generating a full-length genomic (minus strand) HDV RNA standard through in vitro transcription. This standard closely mimics HDV genome RNA purified from virions, serving as a robust calibration tool for reverse transcription and amplification processes during quantification. It is important to note that while theoretically recognized calibration standards were established for HDV RNA quantification, they were not calibrated with the international standard from the World Health Organization (WHO). Further calibration may be necessary in the future to obtain an international unit (IU) readout for this dual detection system.

In the context of HBV DNA quantification within the dual detection system, specific over-gap amplification primers designed for HBV rcDNA have previously been developed. These primers selectively target infectious HBV with rcDNA genomes, excluding non-infectious HBV particles that only possess short minus-strand DNA. This design proves particularly valuable for accurately quantifying the viral load of HBV, especially in samples from patients undergoing Nucleos(t)ide Analog (NAs) therapy.

Moreover, the HBV detection system effectively eliminates the detection of HBV RNA. Prior research has shown that encapsidated and secreted HBV RNA are 3′ terminus truncated, rendering the HBV detection system capable of valid amplification and subsequent quantification exclusively for HBV rcDNA. Consequently, the integration of this HBV rcDNA specific detection into the HBV-HDV dual detection system enhances the specificity of the HBV DNA quantification in this one-step reverse transcription and quantification amplification combined system.

The HBV-HDV dual system is designed to be compatible for pan-genotypic detection of both HBV and HDV. HBV DNA polymerase (pol) lacks proofreading function (3′ exonuclease activity), resulting in remarkable genetic heterogeneity, with at least nine classified genotypes (A to I). HDV, with its high genome variety, exhibits sequence heterogeneity of up to 30% among different genotypes, which are differently distributed across the globe. A pan-genotypic and universal detection method is advantageous for targeting and identifying different genotypes worldwide.

Through phylogenetic analysis and sequence alignment, highly conserved regions for HBV and HDV were identified, allowing the inventors to design primers and probes, respectively. Leveraging the affinity plus probe technology, structural stability was enhanced, increasing the hybridization melt temperature, even with a shorter sequence in limited conserved regions. Notably, in the HDV forward primer, a CIT polymorphism between genotype 4 and other genotypes necessitated the use of a degenerate base Y to ensure the pan-genotypic detection feature for HDV.

The HBV-HDV dual detection system has numerous applications, providing critical insights into interactions between these two viruses. The readouts of HBV DNA and HDV RNA serve as valuable markers for monitoring responses to antiviral therapies, particularly in the current era of anti-HBV spanning approximately two decades. With the approval of bulevirtide heralding a new era of anti-HDV therapy, the dual detection system holds promise in assessing treatment outcomes.

Using the HBV-HDV interaction through this system, its utility in in vitro HBVHDV transfection and infection models was identified. The results indicate that HDV may impede HBV DNA replication and the expression of secreted proteins, consequently increasing intracellular core protein accumulation. Conversely, as expected, HBV appears to facilitate the release of progeny HDV virions while decreasing intracellular HDV antigens.

Upon applying the HBV-HDV dual detection system to in vivo samples, stable levels of HBV DNA and HDV RNA were consistently detected in plasma or serum samples from both human liver chimeric mice and patients. Notably, despite effective NA treatment suppressing HBV DNA to low or undetectable levels, HDV RNA levels remained maintained at high levels.

A number of implementations have been described. Nevertheless, it will be understood that various modifications may be made without departing from the spirit and scope of the disclosure. Accordingly, other implementations are within the scope of the following claims.

1. A method for simultaneous detection and quantification of hepatitis B virus (HBV) DNA and hepatitis delta virus (HDV) RNA in a sample, the method comprising:
combining the sample with an HBV primer pair, an HDV primer pair, an HBV probe, and an HDV probe in a reaction mixture;
performing a one-step reverse transcription quantitative polymerase chain reaction (RT-qPCR) on the reaction mixture; and
detecting a first fluorescent signal from the HBV probe and a second fluorescent signal from the HDV probe to simultaneously quantify HBV DNA and HDV RNA in the sample;
wherein the HBV primer pair comprises an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2];
wherein the HDV primer pair comprises an HDV forward primer having an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4];
wherein the HBV probe comprises a first fluorescent label attached to a 5′ end and a first quencher attached to a 3′ end; and
wherein the HDV probe comprises a second fluorescent label attached to a 5′ end and a second quencher attached to a 3′ end.

2. The method of claim 1, wherein the one-step RT-qPCR is performed in a single reaction tube.

3. The method of claim 1, wherein the first fluorescent label is carboxyfluorescein (FAM) and the second fluorescent label is carboxytetramethylrhodamine (TAMRA).

4. The method of claim 1, wherein the first fluorescent label and the second fluorescent label emit fluorescence at different wavelengths.

5. The method of claim 1, wherein the HBV probe comprises the nucleotide sequence AGATG+ATTA+GGCAGA+GGTGAAAAAG [SEQ ID NO. 5], wherein+denotes a locked nucleic acid modification.

6. The method of claim 1, wherein the HDV probe comprises the nucleotide sequence GGTCC+G+ACCTGGGC+ATCCG [SEQ ID NO. 6], wherein+denotes a locked nucleic acid modification.

7. The method of claim 1, wherein the one-step RT-qPCR comprises:
a reverse transcription step at a temperature between 40 °C and 70 °C for a period of 5 to 20 minutes;
an initial denaturation step at a temperature between 70 °C and 99 °C for a period of less than 5 minutes; and
40 to 60 amplification cycles comprising a denaturation phase and an annealing/extension phase with signal capture.

8. The method of claim 1, wherein the sample comprises a biological sample obtained from a subject.

9. A kit for simultaneous detection and quantification of hepatitis B virus (HBV) DNA and hepatitis delta virus (HDV) RNA, the kit comprising:
an HBV primer pair comprising an HBV forward primer having an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2];
an HDV primer pair comprising an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4];
an HBV probe comprising a first fluorescent label attached to a 5′ end and a first quencher attached to a 3′ end; and
an HDV probe comprising a second fluorescent label attached to a 5′ end and a second quencher attached to a 3′ end;
wherein the first fluorescent label and the second fluorescent label are spectrally distinguishable.

10. The kit of claim 9, wherein the first fluorescent label is carboxyfluorescein (FAM) and the second fluorescent label is carboxytetramethylrhodamine (TAMRA).

11. The kit of claim 9, wherein the HBV probe comprises the nucleotide sequence AGATG+ATTA+GGCAGA+GGTGAAAAAG [SEQ ID NO. 5], wherein+denotes a locked nucleic acid modification.

12. The kit of claim 9, wherein the HDV probe comprises the nucleotide sequence GGTCC+G+ACCTGGGC+ATCCG [SEQ ID NO. 6], wherein+denotes a locked nucleic acid modification.

13. The kit of claim 9, further comprising a reverse transcriptase and a DNA polymerase.

14. A dual fluorescence channel detection system for pan-genotypic simultaneous quantification of hepatitis B virus (HBV) DNA and hepatitis delta virus (HDV) RNA, the system comprising:
a reaction mixture comprising:
an HBV primer pair targeting a conserved region of the HBV genome, the HBV primer pair comprising an HBV forward primer nucleotide sequence GGAGGCTGTAGGCATAAATTGG [SEQ ID NO. 1] and an HBV reverse primer having an HBV reverse primer nucleotide sequence CACAGCTTGGAGGCTTGAAC [SEQ ID NO. 2];
an HDV primer pair targeting a conserved region of the HDV genome, the HDV primer pair comprising an HDV forward primer nucleotide sequence GCGCCGGCYGGGCAAC [SEQ ID NO. 3] and an HDV reverse primer having an HDV reverse primer nucleotide sequence TCCTCTTCGGGTCGGCATG [SEQ ID NO. 4];
an HBV probe comprising a first fluorescent label and a first quencher; and
an HDV probe comprising a second fluorescent label and a second quencher; and
a quantitative PCR instrument configured to detect the first fluorescent label in a first detection channel and the second fluorescent label in a second detection channel.

15. The system of claim 14, wherein the HBV primer pair is configured to specifically detect HBV relaxed circular DNA (rcDNA) through over-gap amplification.


SEQUENCE LISTING (6 sequences)

SEQ ID NO: 1 - DNA, 22 bp, unidentified
  source 1..22: mol_type = unassigned DNA
ggaggctgtaggcataaattgg

SEQ ID NO: 2 - DNA, 20 bp, unidentified
  source 1..20: mol_type = unassigned DNA
cacagcttggaggcttgaac

SEQ ID NO: 3 - DNA, 16 bp, unidentified
  source 1..16: mol_type = unassigned DNA
gcgccggcygggcaac

SEQ ID NO: 4 - DNA, 19 bp, unidentified
  source 1..19: mol_type = unassigned DNA
tcctcttcgggtcggcatg

SEQ ID NO: 5 - DNA, 25 bp, unidentified
  source 1..25: mol_type = unassigned DNA
agatgattaggcagaggtgaaaaag

SEQ ID NO: 6 - DNA, 19 bp, unidentified
  source 1..19: mol_type = unassigned DNA
ggtccgacctgggcatccg